SEQUENCE LISTING (33 sequences)

SEQ ID NO: 1 - protein, 23 aa, Mus musculus
VMGLRILLLKVAGFNLLMTLRLW

SEQ ID NO: 2 - protein, 23 aa, Mus musculus
VMGLRILFLKVFGFSLLMTLRLW

SEQ ID NO: 3 - protein, 23 aa, Mus musculus
TILYEILLGKATLYAVLVSTLVV

SEQ ID NO: 4 - protein, 23 aa, Mus musculus
TILYEILFGKAFLYSVLVSTLVV

SEQ ID NO: 5 - DNA, 33 bp, Artificial Sequence
  synthetic primer
ggatccaatattcagaacccagaacctgctgtg

SEQ ID NO: 6 - DNA, 49 bp, Artificial Sequence
  synthetic primer
cagcgtcatgagcaggctaaatccaaatactttcagaaagaggattcgg

SEQ ID NO: 7 - DNA, 49 bp, Artificial Sequence
  synthetic primer
ccgaatcctctttctgaaagtatttggatttagcctgctcatgacgctg

SEQ ID NO: 8 - DNA, 29 bp, Artificial Sequence
  synthetic primer
ctgcaggcggccgcgtgaggaggacggac

SEQ ID NO: 9 - DNA, 22 bp, Artificial Sequence
  synthetic primer
tctagaggacctgagaaatgtg

SEQ ID NO: 10 - DNA, 25 bp, Artificial Sequence
  synthetic primer
ggtcgactctagaactagtggatcc

SEQ ID NO: 11 - DNA, 34 bp, Artificial Sequence
  synthetic primer
atcctattcgggaaggaaggccttcctatattct

SEQ ID NO: 12 - DNA, 34 bp, Artificial Sequence
  synthetic primer
gcacagaatataggaaggccttcccgaataggat

SEQ ID NO: 13 - protein, 9 aa, Mus musculus
KVPRNQDWL

SEQ ID NO: 14 - protein, 22 aa, Homo sapiens
VIGFRILLLKVAGFNLLMTLRL

SEQ ID NO: 15 - protein, 22 aa, Homo sapiens
VIGFRILFLKVFGFSLLMTLRL

SEQ ID NO: 16 - protein, 22 aa, Homo sapiens
VMGFRILFLKVFGFSLLMTLRL

SEQ ID NO: 17 - protein, 23 aa, Homo sapiens
TILYEILLGKATLYAVLVSALVL

SEQ ID NO: 18 - protein, 23 aa, Homo sapiens
TILYEILFGKAFLYSVLVSALVL

SEQ ID NO: 19 - protein, 23 aa, Mus musculus
TILYEILLGKATLYAVLVSGLVL

SEQ ID NO: 20 - protein, 23 aa, Mus musculus
TILYEILFGKAFLYSVLVSGLVL

SEQ ID NO: 21 - DNA, 69 bp, Mus musculus
gtgatgggcctgagaatcctgctgctgaaggtggccggcttcaacctgctgatgaccctgaggctgtgg

SEQ ID NO: 22 - DNA, 69 bp, Mus musculus
gtgatgggcctgagaatcctgttcctgaaggtgttcggcttcagcctgctgatgaccctgaggctgtgg

SEQ ID NO: 23 - DNA, 69 bp, Mus musculus
accatcctgtacgagatcctgctgggcaaggccacactgtacgccgtgctggtgtccggcctggtgctg

SEQ ID NO: 24 - DNA, 69 bp, Mus musculus
accatcctgtacgagatcctgttcggcaaggccttcctgtactctgtgctggtgtccggcctggtgctg

SEQ ID NO: 25 - DNA, 66 bp, Homo sapiens
gtgattgggttccgaatcctcctcctgaaagtggccgggtttaatctgctcatgacgctgcggctg

SEQ ID NO: 26 - DNA, 66 bp, Homo sapiens
gtgattgggttccgaatcctctttctgaaagtgtttgggttttccctgctcatgacgctgcggctg

SEQ ID NO: 27 - DNA, 66 bp, Homo sapiens
gtgatggggttccgaatcctctttctgaaagtgtttgggttttccctgctcatgacgctgcggctg

SEQ ID NO: 28 - DNA, 69 bp, Homo sapiens
accatcctctatgagatcttgctagggaaggccaccttgtatgccgtgctggtcagtgccctcgtgctg

SEQ ID NO: 29 - DNA, 69 bp, Homo sapiens
accatcctctatgagatcttgtttgggaaggcctttttgtattccgtgctggtcagtgccctcgtgctg

SEQ ID NO: 30 - protein, 12 aa, Homo sapiens
  MISC_FEATURE (2)..(4): X may be any amino acid
  MISC_FEATURE (6)..(7): X may be any amino acid
  MISC_FEATURE (9)..(11): X may be any amino acid
FXXXFXXSXXXS

SEQ ID NO: 31 - protein, 12 aa, Homo sapiens
  MISC_FEATURE (2)..(4): X may be any amino acid
  MISC_FEATURE (6)..(7): X may be any amino acid
  MISC_FEATURE (9)..(11): X may be any amino acid residue
  MISC_FEATURE (9)..(11): X may be any amino acid
FXXXFXXTXXXS

SEQ ID NO: 32 - protein, 8 aa, Mus musculus
SIINKFEL

SEQ ID NO: 33 - protein, 9 aa, Homo sapiens
YLEPGPVTA

(19) United States
(12) Patent Application Publication
(10) Pub. No.: US 2019/0030071 A1
(43) Pub. Date: Jan. 31, 2019

(54) T CELL RECEPTORS

(71) Applicant: IMMURES S.r.l., Roma (IT)

(72) Inventors: Piergiuseppe de Berardinis, Roma (IT); Luciana d'Apice, Roma (IT); Umberto Oreste, Roma (IT)

(73) Assignee: IMMURES S.r.I., Roma (IT)

(21) Appl. No.: 15/523,925

(22) PCT Filed: Nov. 3, 2015
(86) PCT No.: PCT/EP2015/075607

§ 371 Date: May 2, 2017

(30) Foreign Application Priority Data
Nov. 3, 2014 (IT) RM2014A000632
Aug. 13, 2015 (IT) 102015000044533

(51) Int. Cl. A61K 35/17; C12N 5/0783; C07K 14/725; A61K 38/17; A61K 39/00; C12N 5/078

(52) U.S. Cl. CPC A61K 35/17; C12N 5/0636; C07K 14/7051; A61K 38/177; C12N 2501/2302; C12N 2502/11; C12N 5/0634; A61K 39/0011; C12N 2501/24; C12N 2501/515; A61K 2039/5156

(57) ABSTRACT

The present invention relates to modified T cell receptors (TCRs) and to their use in adoptive cell therapy (ACT), in particular for the transfer of T lymphocytes. The TCRs are mutated in the transmembrane regions of the alpha and beta chains with mutations favoring the correct TCR chain pairing. The correct pairing of the transferred exogenous alpha and beta TCR chains improves the functional activity and safety of the genetically modified T cells for the therapy of tumours and infectious diseases. The invention also relates to T cell receptor alpha or beta chain, to a recombinant TCR, a TCR complex, a nucleic acid coding for the TCR alpha or beta chain, to relative recombinant expression vector, host cells, pharmaceutical composition and to a method of detecting a hematological malignant cell, a solid tumor cell or an infected cell.

### FIELD OF THE INVENTION

The present invention relates to modified T cell receptors (TCRs) and to their use in adoptive cell therapy (ACT), in particular for the transfer of T lymphocytes. The TCRs are mutated in the transmembrane regions of the alpha and beta chains with mutations favoring the correct TCR chain pairing. The correct pairing of the transferred exogenous alpha and beta TCR chains improves the functional activity and safety of the genetically modified T cells for the therapy of tumours and infectious diseases. The invention also relates to T cell receptor alpha or beta chain, to a recombinant TCR, a TCR complex, a nucleic acid coding for the TCR alpha or beta chain, to relative recombinant expression vector, host cells, pharmaceutical composition and to a method of detecting a hematological malignant cell, a solid tumor cell or an infected cell.

### BACKGROUND OF THE INVENTION

Adoptive cell therapy (ACT), in particular for the transfer of T lymphocytes has proven to be an effective therapeutic strategy against tumours and infectious diseases. Since the isolation and in vitro expansion of the required number of antigen specific T cells is difficult, a strategy to transfer the genes encoding T cell receptor (TCR) of known specificity has been described and applied in several trials. It is possible to engineer T lymphocytes to express human TCRs that confer novel specificities against tumours and infectious pathogens. Recently, this approach has been proved clinically beneficial as the transfer of such “reprogrammed” cells to terminally ill cancer patients led to tumour regression in a significant percentage of treated patients (1).

The international patent application WO2007017201 is directed to a method of generating antigen specific T cells, to the antigen specific T cells thereof, the isolated transgenic TCR's and to pharmaceutical compositions containing the same and their use in adoptive cell therapy. In particular, the document pertains to the use of cells co-expressing allogenic MHC molecules and antigens to induce peptide-specific T cells from non-selected allogenic T cell repertoires. The international patent application WO2011001152 refers to a T cell receptor having the property of binding to the gp100₂₈₀₋₂₈₈ YLEPGPVTA (SEQ ID No: 33) peptide-HLA-A2 complex and comprising a TCR alpha variable domain and/or a TCR beta variable domain characterized in that said TCR is mutated. The document also refers to the use of such TCRs in adoptive therapy and fusions of such TCRs with therapeutic agents.

The international patent application WO2014083173 relates to a method for the production of novel T cell receptors which provide a reduced risk of adverse events in immune therapy, specifically in adoptive T cell transfer. Such method comprises the following steps: a) providing a host organism expressing un-rearranged human TCR loci, b) immunizing said host organism with a peptide comprising an epitope specific for a tumor specific antigen (TSA), c) isolating from said host organism a T cell clone having an activity against said human mutated TSA, d) optionally, isolating from said T cell clone the TCR, wherein said TSA is selected out of the class of somatic mutated antigens. The TCRs produced according to the disclosed are specific for tumor cells and do not react with healthy tissue.

The international patent application WO2008071701 is directed to LAK-T cells which have been transformed by a transgenic T cell receptor, to a method of generating those transgenic T cells and to a pharmaceutical composition comprising said cells and the use of the LAK-T cells or of the pharmaceutical composition in the adoptive cell therapy and for treating hematological malignancies or solid tumors or acute or chronic infections or autoimmune diseases.

Although promising, several hurdles, including the proper expression of the exogenous TCR, have hampered the clinical impact of this ACT approach. Clinical responses indicate that many problems are still to be solved. As T cell functional avidity is dictated mainly by both TCR affinity and the number of TCR molecules expressed [14], much efforts have been devoted to improving these biophysical properties in TCR-engineered cells using two important approaches: (a) the improvement of TCR affinity and (b) the enhancement of TCR expression. To improve TCR affinity, attempts have been made to select high affinity receptors [15] or to enhance the affinity of the transferred receptor by point mutations [16]. Alternatively, various approaches have been devised to increase the number of TCRs on the surface of transduced cells. These include engineering expression vectors, using of codon-optimized TCR sequences, eliminating glycosylation sites and improving pairing of the introduced TCR chains.

Haga-Friedman et al [4] report an approach to enhance surface expression of TCR chains to improve cellular avidity and anti-tumor TCR activity. Exogenous TCR stability is improved by increasing the hydrophobic nature of TCRα transmembrane region. Selected residues were changed to either leucine or valine on the TCRα-chain. The document reports that even a small number of mutations in the TCRβ-chain were found to abrogate almost completely TCR expression and function.

Many studies focus on enhancing the preferential pairing of transferred TCRαβ combinations. Indeed, it is known that one of the major limitation for ACT is the formation of mispaired TCR components which may reduce the efficacy of the genetically modified primary T cells or may cause autoimmune damages when transferred into patients. When another exogenous TCR is introduced by gene transfer in primary T cells, the endogenous alpha and beta chains of the TCR can cross pair with the reciprocal transgenic chains to produce a new hybrid TCR. Thus, four different αβ dimers can form in the transduced cells: the endogenous TCR (α_{EN}/β_{EN}), the transduced TCR (α_{TR}/β_{TR}) and two unproductive mixed dimers (α_{EN}/β_{TR} and α_{TR}/β_{EN}). Since the surface expression of these TCR necessitates the assembly with a limited number of CD3 molecules, the existence of unproductive forms of TCR leads to reduced levels of the exogenous TCR. Additionally, recent reports showed that these mixed TCRs heterodimers might engender autoimmunity manifestations and self-reactivity (Graft-versus-host disease, GVHD) both in vitro and in pre-clinical models [2].

Therefore, many groups have devised strategies to reduce the mispairing effect as well as to promote the correct pairing of the exogenous TCR chains. Such strategies were reported in the recent years [3], and include:
The addition of a second disulphide bond by introducing cysteine residues by single point mutations [18];
The “murinization” of all or part of the TCR constant regions by swapping the human C region with the murine one or by introducing essential murine residues that mediate a preferential TCR-chain pairing (for instance by single point mutations) [19];
The use of chimericTCR-CD3ζ chain having each TCR chain fused to a CD3ζ molecule [20] or of single-chain TCRs wherein the Vα of a defined TCR is fused to the beta chain using a flexible peptide linker [21];
The use of shRNA sequences or zinc finger nucleases to knock down the expression of the endogenous TCR [22].

Another approach was proposed by O'Shea et al [5] who designed a pair of peptides, named velcro, able to pair with one another due to favourable electrostatic interactions in the heterodimeric state. These authors demonstrated that the two peptides are predominantly unfolded in isolated form but associate preferentially to form a stable parallel, coiled coil heterodimer when mixed. This approach was also applied by Chang et al [6] to produce soluble TCR in which heterodimeric complex was favoured by fusing the peptides to truncated alpha and beta chains respectively.

After several years of research in this field there is still the need to find new easy methods favouring correct TCR pairing that can be applied to clinical treatments. All reported approaches suffer some limitations due to low affinity and specificity of the new receptors, or to the need of in vitro manipulations which reduce the best fitness of the transduced T cells. Therefore it is a problem underlying the present invention to provide a strategy for rapid and effective generation of TCRs wherein the alpha and beta chain are properly associated, thus resulting into high target affinity and low immunogenic potential.

The authors of the present invention have focused their efforts in developing a methodology requiring minimal manipulation of TCR chains and host T cells.

### SUMMARY OF THE INVENTION

Recently a FXXXFXXSXXXS (SEQ ID No: 30) or a FXXXFXXTXXXS (SEQ ID No: 31) motif was identified in the transmembrane region of the membrane bound form of the immunoglobulin heavy chain [7]. This motif is evolutionary conserved in very distant species (from shark to mammals) and was found by using lipid bilayer molecular dynamics simulations. The motif is responsible for the two heavy chains association through two pairs of Phe-Phe hydrophobic interactions and two pairs of Ser/Thr-Ser/Ser hydrogen bonds. This interaction pattern, which stabilizes the dimer conformation in the lipid bilayer, is unique. The pattern is different from any other pattern identified in transmembrane helices and has been demonstrated to possess a number of interesting structural implications in the immunoglobulin heavy chain (IgTMD) dimer assembly and, in turn, in the function of the B-cell receptor (BCR).

The object of the invention is to solve one of the major limitation of adoptive cell therapy based on the use of genetically modified T cells: the formation of mispaired TCR that can affect the efficacy of the genetically modified T cells or determine autoimmune damages when they are transferred into the patients. It was surprisingly found that a motif as above indicated, once inserted into the transmembrane constant region of both alpha and beta chains of TCRs, provides preferential pairing of exogenous TCR chains. In particular, the present invention provides alpha and beta chains wherein at least three critical amino acids in both chains are mutated into two hydrophobic and one polar amino acids. Two hydrophobic and one polar residues provide assistance in the correct pairing, by forming respectively two interhelical 7E-7E interactions and one hydrogen bond. This rather unexpected finding provides an innovative solution to prevent the mispairing of TCR chains and to ultimately achieve a higher functional avidity of TCR gene modified T cells.

It has been demonstrated by the present invention that the mutated TCR chains according to the invention are correctly assembled and transported to the cell membrane. For TCR export to membrane the correct pairing of alpha and beta chain and binding to CD3 complex are both necessary [9]. Both alpha and beta TCR chains participate in binding to CD3 complex since TCR alpha chain can assemble with CD3 delta and zeta chains but not CD3 gamma and zeta. In contrast, the TCR beta chain can assemble with any of the CD3 chains except the zeta chain.

For these reasons it is not observed the formation of alpha and beta homodimers of the mutated chains of the present invention.

The TCRs modified as described herein have been produced and introduced into a hybridoma cell line. The mutated TCRs block TCR mispairing, not affecting TCR function and are safe to the host cell system.

The present technology requires minimal intervention and manipulation on the native primary structure of alpha and beta chain. The present technology does not require the introduction of further intracellular or extracellular sites of interchains bindings or further actions on the recipient primary T cells. It does not displays new non-self antigenic epitopes. Many of the modifications proposed by others require the introduction of sequences from non-human origin (i. e. murinization) and these sequences are recognised as non self by the immune system and could start an immune response against the modified T cells carrying this TCR (GVHD, graft-versus-host disease [2]).

The skilled in the art can easily develop the construction of purposely tailored cassette vectors for rapid cloning and expression of TM mutated alpha and beta TCR chains. The invention is applicable to any TCR with different specificity and can replace the TCR currently used in cancer therapy to avoid side effects of reduced specificity and potential GVHD (Graft-versus-host disease).

Further, the mutated chains were introduced in isolated mouse spleen cells and the results demonstrate that mutated TCR maintains specificity of TCR recognition against a selected antigen, confirming therapeutic efficacy in a murine system.

In view of the state of the art, the present invention provides a novel strategy to design TCR alpha and beta chains carrying reciprocal mutations that sustain the steric and electrostatic interactions at the interface of the constant domain such that almost exclusively the exogenous TCRs chains complementary fit together, thereby avoiding the formation of hybrid TCRs. The authors of the present invention developed a methodology with the aim to promote the correct pairing of the transferred exogenous alpha and beta TCR chains thus improving the functional activity and safety of T cells genetically modified with such TCR. The proposed invention consists in modifying the transmembrane regions of alpha and beta TCR chains by introducing amino acidic mutations driving a pairing specific for the exogenous alpha and beta chains. This is achieved by applying the motif previously identified.

It was surprisingly found that a motif comprising two hydrophobic and one polar amino acid residues, once inserted into the transmembrane domain of a TCR, improves TCR functionalities. The mutated TCR chains have been demonstrated to be effective in transducing primary T cells, conferring them the required antigen specificity. The mispairing between exogenous and endogenous alpha and beta TCR chains is blocked. High expression of functional exogenous TCR leads to increased activity.

Then the present invention provides a T Cell Receptor (TCR) alpha chain comprising a transmembrane region comprising SEQ ID NO:1 or SEQ ID NO:14, or a T Cell Receptor (TCR) beta chain comprising a transmembrane region comprising SEQ ID NO:3, SEQ ID NO:17 or SEQ ID NO:19 wherein said region is characterized in that it is mutated in the amino acid positions 8 and 12 with an hydrophobic amino acid residue, and in position 15 with a polar amino acid residue.

Preferably the hydrophobic amino acid residue is phenylalanine and the polar amino acid residue is serine or threonine.

Still preferably the T Cell Receptor (TCR) alpha chain is further mutated in position 2 with the amino acid methionine.

In a preferred form the polar aminoacid is serine.

In a preferred embodiment the alpha chain comprises a sequence selected from SEQ ID NO:2 or SEQ ID NO:15 and the beta chain comprises a sequence selected from SEQ ID NO:4, SEQ ID NO:18 or SEQ ID NO:20.

Preferably the T Cell Receptor (TCR) alpha chain comprises SEQ ID NO:16.

The present invention provides an isolated and/or recombinantly engineered T Cell Receptor (TCR), characterized by comprising the alpha chain and the beta chain as described above.

Preferably in the T Cell Receptor (TCR), the transmembrane region of the alpha chain comprises SEQ ID NO:2 and the transmembrane region of the beta chain comprises SEQ ID NO:4 or SEQ ID NO:20.

Preferably the transmembrane region of the alpha chain comprises SEQ ID NO:15 and the transmembrane region of the beta chain comprises SEQ ID NO:18.

Still preferably the transmembrane region of the alpha chain comprises SEQ ID NO:16 and the transmembrane region of the beta chain comprises SEQ ID NO:18.

Preferably the T Cell Receptor (TCR) is specific for an antigen selected from the group consisting of: a tumor cell antigen, a tumor cell associated antigen, a pathogenic agent, preferably said agent derives from a virus, bacteria, protozoa or a parasite.

Still preferably the tumor cell antigen or the tumor cell associated antigen is selected from an antigen of a hematological malignancy or of a solid tumor, preferably multiple myeloma, melanoma, lung tumor, endometrial tumors, glioma, lymphoma, leukemia or prostate tumor.

More preferably the T Cell Receptor (TCR) of the invention is selective for a virus selected from the group consisting of: influenza virus, measles and respiratory syncytial virus, dengue virus, human immunodeficiency virus, human hepatitis virus, herpes virus, papilloma virus or for a protozoa that is Plasmodium falciparum or for a bacteria that is a mycobacteria causing tuberculosis.

Preferably the T Cell Receptor (TCR) is associated with a detectable label, a therapeutic agent, a pharmacokinetic modifying moiety or a combination of any of these.

In particular the therapeutic agent is an anti-tumoral agent, an antibiotic, an anti-infectious disease agent.

The present invention also provides a TCR complex comprising at least two TCRs as defined above.

The present invention also provides a nucleic acid coding for the T Cell Receptor (TCR) alpha and/or beta chain according to the invention or for the T Cell Receptor (TCR) according to the invention.

The present invention also provides a recombinant expression vector comprising the nucleic acid of the invention, said vector being preferably a retroviral or lentiviral vector.

The present invention also provides a host cell transformed, transduced or transfected with the recombinant expression vector as defined above, or comprising the nucleic acid as defined above, preferably said host cell is a T lymphocyte.

The present invention also provides a method to generate cells expressing the T Cell Receptor (TCR) as defined above comprising the following steps:

a) Activating a population of lymphocytes obtained from peripheral blood of a subject;

b) Isolating the T cells from said population;

c) Transducing or transfecting the isolated T cells with a nucleic acid as defined above, preferably said nucleic acid is comprised in a vector.

The present invention also provides a cell expressing the T Cell Receptor (TCR) which is obtainable by the method described above.

The present invention also provides a pharmaceutical composition comprising the nucleic acid of the invention, the vector of the invention or the cell of the invention and pharmaceutically acceptable vehicle and/or adjuvant.

Preferably the T Cell Receptor (TCR), the TCR complex, the nucleic acid, the vector or the cell of the invention are for use as a medicament, preferably for use in adoptive cell therapy, preferably a T lymphocyte adoptive transfer, still preferably for use in the treatment and/or prevention of a hematological malignancy, a solid tumor or an infective disease.

The present invention also provides a method for the treatment and/or prevention of a hematological tumor, a solid tumor or an infective disease comprising administering in an effective amount the T Cell Receptor (TCR), the TCR complex, the nucleic acid, the vector or the cell of the invention in a subject in need thereof.

The present invention also provides a method of detecting a hematological malignant cell, a solid tumor cell or an infected cell comprising:

(i) contacting a sample comprising the hematological malignant cell, solid tumor cell or the infected cell with the T Cell Receptor (TCR), the TCR complex, the nucleic acid, the vector or the cell of the invention as described above, thereby forming a complex, and (ii) detecting the complex, wherein detection of the complex is indicative of the presence of an hematological malignancy, a solid tumor or an infective disease.

The invention further provides related polypeptides and proteins, as well as related nucleic acids, recombinant expression vectors, host cells, and populations of cells. Further provided by the invention are antibodies, or antigen binding portions thereof, and pharmaceutical compositions relating to the TCRs (including functional portions and functional variants thereof) of the invention.

Methods of detecting a hematopoietic malignant cell or a solid tumor cell or an infected cell and methods of treating or preventing a hematological tumor, or a solid tumor or an infectious disease in a mammal are further provided by the invention. The inventive method of detecting a hematopoietic malignant or tumor cell or infected cell comprises (i) contacting a sample comprising leukemia cells with any of the inventive TCRs (including functional portions and functional variants thereof), polypeptides, proteins, nucleic acids, recombinant expression vectors, host cells, populations of host cells, or antibodies, or antigen binding portions thereof, described herein, thereby forming a complex, and (ii) detecting the complex, wherein detection of the complex is indicative of the presence of a hematological malignancy or a solid tumor or an infective disease. The inventive method of treating or preventing a hematological tumor, a solid tumor or an infective disease in a mammal comprises administering to the mammal any of the TCRs (including functional portions and functional variants thereof), polypeptides, or proteins described herein, any nucleic acid or recombinant expression vector comprising a nucleotide sequence encoding any of the TCRs (including functional portions and functional variants thereof), polypeptides, proteins described herein, or any host cell or population of host cells comprising a recombinant vector which encodes any of the TCRs (including functional portions and functional variants thereof), polypeptides, or proteins described herein, in an amount effective to treat or prevent the α hematological tumor, a solid tumor or an infective disease in the mammal.

The invention provides an isolated or purified T cell receptor (TCR), and functional portions and functional variants thereof, having improved pairing properties.

The TCRs (including functional portions and functional variants thereof) of the invention provide many advantages, including when used for adoptive cell transfer or therapy. For example, by having an improved pairing property, the inventive TCRs (including functional portions and functional variants thereof) make it possible to target the destruction of cancer cells or infected cells while minimizing or eliminating the destruction of normal, non-cancerous cells, thereby reducing, for example, by minimizing or eliminating, toxicity and minimizing GVHD.

The inventive TCRs (including functional portions and functional variants thereof) advantageously provide the ability to destroy specifically hematopoietic cells, solid tumor cells and infective cells and accordingly provide the ability to treat or prevent a hematological tumor, a solid tumor or an infective disease. Additionally, without being bound by a particular theory, it is believed that because of the reduction of mixed dimers formation the inventive TCR reduces the off target recognition and the risk of GVHD (Lethal graft-versus-host disease) in mouse models of T cell receptor gene therapy [23] and increase the ability of T cell carrying the described TCR to recognize the specific antigen. Accordingly, the inventive TCRs (including functional portions and functional variants thereof) advantageously greatly expand the patient population that can be treated.

The invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has improved pairing properties.

Within the meaning of the present invention, the terms “peptide” or “polypeptide” are not particularly restricted, and in general designate natural or synthetic peptides containing only natural amino acids, only non-natural amino acids, or combinations of natural and non-natural amino acids. In the context of the present invention, the term “peptide” denotes a chain of amino acids linked together via a peptide bond (or amide bond). The term “amino acid” as employed herein includes and encompasses all of the naturally occurring amino acids, either in the D-, L-, allo, or other stereoisomeric configurations if optically active, as well as any known or conceivable non-natural, synthetic and modified amino acid.

The term “natural amino acids” denotes the following 20 amino acids in their laevorotatory (L) or dextrorotatory (D) form, preferably in their natural L form:

| Name | One-letter code | Three-letter code |
|---|---|---|
| Alanine | A | Ala |
| Arginine | R | Arg |
| Asparagine | N | Asn |
| Aspartate | D | Asp |
| Cysteine | C | Cys |
| Glutamate | E | Glu |
| Glutamine | Q | Gln |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

The term “hydrophobic” amino acid denotes one of the following amino acids: Alanine, Isoleucine, Leucine, Phenylalanine, Valine, Proline, Glycine. The term “polar” amino acid denotes one of the following amino acids: Glutamine, Asparagine, Histidine, Serine, Threonine, Tyrosine, Cysteine, Methionine, Tryptophan. The term “charged” amino acid denotes one of the following amino acids: Arginine, Lysine, Aspartic acid, Glutamic acid.

Within the meaning of the present invention, the mutations introduced into the alpha and beta chain of a TCR comprise at least two hydrophobic and one polar amino acid residues.

The polynucleotide and/or peptides of the invention can comprise one or more conservative substitutions. The term “conservative substitutions” as used herein include nucleotide substitutions that do not result in changes in the amino acid sequence, as well as nucleotide substitutions that result in conservative amino acid substitutions, or amino acid substitutions which do not substantially affect the character of the polypeptide translated from said nucleotides.

Conservative substitutions of amino acid sequences include amino acid substitutions or deletions that do not substantially affect the character of the variant polypeptide relative to the starting peptide. For example, polypeptide character is not substantially affected if the substitutions or deletions do not preclude specific binding of the variant peptide to a specific binding partner of the starting peptide. Included in this definition are amino acid insertions, substitutions, deletions and truncations that do not substantially affect the polypeptide character relative to the starting peptide. Included also are other variants and derivatives that will be apparent to those skilled in the art and are considered to fall within the scope of this invention. In an embodiment, the conservative substitution is selected from the following six groups which each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

As used herein, the term “orthologous” refers to protein in species different with respect to the proteins of SEQ ID NO:14 and SEQ ID NO:17 in Homo Sapiens and SEQ ID NO:1 and SEQ ID NO:3 and SEQ ID NO:19 in Mus Musculus. As an example of said orthologous, the corresponding proteins in Rattus norvegicus, Gallus gallus, Xenopus laevis and Danio rerio can be cited.

In an embodiment of the invention, the TCR comprises two polypeptide chains, each of which comprises a variable region comprising a complementarity determining region (CDR) CDR1, a CDR2, and a CDR3 of a TCR. In an embodiment of the invention, the TCR comprises a first polypeptide chain comprising a CDR1 (CDR1 of α chain), a CDR2 (CDR2 of α chain) and a CDR3 (CDR3 of α chain) CDR sequences are underlined in the TCR sequences below, and a second polypeptide chain comprising a CDR1 (CDR1 of β chain), a CDR2 (CDR2 of β chain), and a CDR3 (CDR3 of β chain).

Alternatively or additionally, the TCR can comprise an α chain of a TCR and a β chain of a

TCR. Each of the α chain and β chain of the inventive TCR can independently comprise any amino acid sequence. Preferably, the α chain comprises the variable region of an α chain as set forth above. An α chain of this type can be paired with any β chain of a TCR. Preferably, the β chain of the inventive TCR comprises the variable region of a β chain as set forth above. Included in the scope of the invention are functional variants of the inventive TCRs described herein. The term “functional variant” as used herein refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to antigen for which the parent TCR has antigenic specificity or to which the parent polypeptide or protein specifically binds, to a similar extent, the same extent, or to a higher extent, as the parent TCR, polypeptide, or protein. The functional variant improves exogenous alpha and beta TCR chain pairing while eliminating or minimizing unproductive mixed dimers (endogenous/transduced dimers). In reference to the parent TCR, polypeptide, or protein, the functional variant can, for instance, be at least about 30%, 50%, 75%, 80%, 90%, 95%, 96%, 97%, 98%, 99% or more identical in amino acid sequence to the parent TCR, polypeptide, or protein.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, He, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

Like the TCRs of the invention, the functional variants described herein comprise two polypeptide chains, each of which comprises a variable region comprising a CDR1, a CDR2, and a CDR3 of a TCR.

Alternatively or additionally, the functional variant of a TCR can comprise a substituted amino acid sequence of a variable region of a TCR comprising the CDRs.

Alternatively or additionally, the functional variant of a TCR can comprise a substituted a chain of a TCR and a β chain of a TCR. Each of the α chain and β chain of the inventive TCR can independently comprise any amino acid sequence. Preferably, the substituted a chain comprises a substituted variable region of an α chain as set forth above.

In another embodiment of the invention, the TCR (or functional variant thereof) can comprise a human/mouse chimeric TCR (or functional variant thereof). In this regard, the TCR (or functional variant thereof) can comprise a mouse constant region.

The inventive human/mouse chimeric TCR (or functional variant or functional portion thereof) can comprise any of the CDRs.

Alternatively or additionally, the human/mouse chimeric TCR (or functional variant or functional portion thereof) can comprise any of the variable regions set forth above. In this regard, the inventive human/mouse chimeric TCR (or functional variant or functional portion thereof) can comprise the substituted amino acid sequence of the substituted variable region of an α chain, the variable region of a β chain, the substituted amino acid sequence of the substituted variable region of a β chain, the variable region of an α chain. Alternatively or additionally, the human/mouse chimeric TCR (or functional variant or functional portion thereof) can comprise an α chain of a TCR (or functional variant or functional portion thereof) and a β chain of a TCR (or functional variant or functional portion thereof). Each of the α chain and β chain of the inventive human/mouse chimeric TCR (or functional variant or functional portion thereof) can independently comprise any amino acid sequence. Preferably, the α chain comprises the variable region of an α chain as set forth above. In this regard, the inventive human/mouse chimeric TCR (or functional variant or functional portion thereof) can comprise the amino acid sequence of reported below. An inventive human/mouse chimeric TCR (or functional variant or functional portion thereof) of this type can be paired with any β chain of a TCR (or functional variant or functional portion thereof). Preferably, the β chain of the inventive human/mouse chimeric TCR (or functional variant or functional portion thereof) comprises the variable region of a β chain. In this regard, the inventive human/mouse chimeric TCR (or functional variant or functional portion thereof) can comprise the amino acid sequence as reported below. The inventive human/mouse chimeric TCR (or functional variant or functional portion thereof), therefore, can comprise the amino acid sequence of SEQ ID NO: 1-4 and 14-20 or a combination thereof.

Also provided by the invention is a polypeptide comprising a functional portion of any of the TCRs or functional variants described herein. The term “polypeptide” as used herein includes oligopeptides and refers to a single chain of amino acids connected by one or more peptide bonds.

With respect to the inventive polypeptides, the functional portion can be any portion comprising contiguous amino acids of the TCR (or functional variant thereof) of which it is a part, provided that the functional portion specifically binds to antigen, as the parent TCR. The functional variant improves exogenous alpha and beta TCR chain pairing while eliminating or minimizing unproductive mixed dimers (endogenous/transduced dimers). The term “functional portion” when used in reference to a TCR (or functional variant thereof) refers to any part or fragment of the TCR (or functional variant thereof) of the invention, which part or fragment retains the biological activity of the TCR (or functional variant thereof) of which it is a part (the parent TCR or parent functional variant thereof). Functional portions encompass, for example, those parts of a TCR (or functional variant thereof) that retain the ability to specifically bind to an antigen or detect, treat, or prevent a hematological tumor, a solid tumor or an infective disease disorder, to a similar extent, the same extent, or to a higher extent, as the parent TCR (or functional variant thereof). In reference to the parent TCR (or functional variant thereof), the functional portion can comprise, for instance, about 10%, 25%, 30%, 50%, 68%, 80%, 90%, 95%, or more, of the parent TCR (or functional variant thereof). The functional portion can comprise additional amino acids at the amino or carboxy terminus of the portion, or at both termini, which additional amino acids are not found in the amino acid sequence of the parent TCR or functional variant thereof. Desirably, the additional amino acids do not interfere with the biological function of the functional portion, e.g., specifically binding to an antigen; and/or having the ability to detect a hematological tumor, a solid tumor or an infective disease, treat or prevent a hematological tumor, a solid tumor or an infective disease, etc. More desirably, the additional amino acids enhance the biological activity, as compared to the biological activity of the parent TCR or functional variant thereof.

The polypeptide can comprise a functional portion of either or both of the α and β chains of the TCRs or functional variant thereof of the invention, such as a functional portion comprising one of more of CDR1, CDR2, and CDR3 of the variable region(s) of the α chain and/or β chain of a TCR or functional variant thereof of the invention. In this regard, the polypeptide can comprise a functional portion comprising the amino acid sequence of CDR1 of a chain, CDR2 of a chain, CDR3 of a chain, CDR1 of β chain, CDR2 of β chain and CDR3 of β chain, or a combination thereof. Alternatively or additionally, the inventive polypeptide can comprise, for instance, the variable region of the inventive TCR or functional variant thereof comprising a combination of the CDR regions set forth above.

Alternatively or additionally, the inventive polypeptide can comprise the entire length of an α or β chain of one of the TCRs or functional variant thereof described herein. In this regard, the inventive polypeptide can comprise an amino acid sequence of SEQ ID NOs: 1-4 and 14-18. Alternatively, the polypeptide of the invention can comprise α and β chains of the TCRs or functional variants thereof described herein.

The invention further provides a protein comprising at least one of the polypeptides described herein. By “protein” is meant a molecule comprising one or more polypeptide chains.

In an embodiment, the protein of the invention can comprise a first polypeptide chain comprising the amino acid sequences of SEQ ID NOs: 1, 2, 14, 15, 16 and a second polypeptide chain comprising the amino acid sequence of SEQ ID NOs: 3, 4, 17, 18, 19, 20. In this regard, the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, TCR constant gamma, TCR constant delta, CD3, CD28, CD137, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods. See, for instance, Choi et al., Mol. Biotechnol. 31 : 193-202 (2005).

In some embodiments of the invention, the TCRs (and functional portions and functional variants thereof), polypeptides, and proteins of the invention may be expressed as a single protein comprising a linker peptide linking the α chain and the β chain. In this regard, the TCRs (and functional variants and functional portions thereof), polypeptides, and proteins of the invention comprising SEQ ID NO: 1-4, 14-20 may further comprise a linker peptide. The linker peptide may advantageously facilitate the expression of a recombinant TCR (including functional portions and functional variants thereof), polypeptide, and/or protein in a host cell. Upon expression of the construct including the linker peptide by a host cell, the linker peptide may be cleaved, resulting in separated α and β chains.

The protein of the invention can be a recombinant antibody comprising at least one of the inventive polypeptides described herein. As used herein, “recombinant antibody” refers to a recombinant (e.g., genetically engineered) protein comprising at least one of the polypeptides of the invention and a polypeptide chain of an antibody, or a portion thereof. The polypeptide of an antibody, or portion thereof, can be a heavy chain, a light chain, a variable or constant region of a heavy or light chain, a single chain variable fragment (scFv), or an Fc, Fab, or F(ab) 2′ fragment of an antibody, etc. The polypeptide chain of an antibody, or portion thereof, can exist as a separate polypeptide of the recombinant antibody.

Alternatively, the polypeptide chain of an antibody, or portion thereof, can exist as a polypeptide, which is expressed in frame (in tandem) with the polypeptide of the invention. The polypeptide of an antibody, or portion thereof, can be a polypeptide of any antibody or any antibody fragment, including any of the antibodies and antibody fragments described herein. The TCR (or functional variant thereof), polypeptide, or protein can consist essentially of the specified amino acid sequence or sequences described herein, such that other components of the TCR (or functional variant thereof), polypeptide, or protein, e.g., other amino acids, do not materially change the biological activity of the TCR (or functional variant thereof), polypeptide, or protein. In this regard, the inventive TCR (or functional variant thereof), polypeptide, or protein can, for example, consist essentially of the amino acid sequence of SEQ ID NO: 1-4, 14-20.

The TCRs, polypeptides, and proteins of the invention (including functional variants thereof) can be of any length, i.e., can comprise any number of amino acids, provided that the TCRs, polypeptides, or proteins (or functional variants thereof) retain their biological activity, e.g., the ability to specifically bind to antigens; detect a hematological tumor, a solid tumor or an infective disease in a mammal; or treat or prevent a hematological tumor, a solid tumor or an infective disease in a mammal, etc. For example, the polypeptide can be in the range of from about 50 to about 5000 amino acids long, such as 50, 70, 75, 100, 125, 150, 175, 200, 300, 400, 500, 600, 700, 800, 900, 1000 or more amino acids in length. In this regard, the polypeptides of the invention also include oligopeptides.

The TCRs, polypeptides, and proteins of the invention (including functional variants thereof) of the invention can comprise synthetic amino acids in place of one or more naturally-occurring amino acids. Such synthetic amino acids are known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, oc-aminocyclopentane carboxylic acid, a-aminocyclohexane carboxylic acid, a-aminocycloheptane carboxylic acid, a-(2-amino-2-norbornane)-carboxylic acid, α,γ-diaminobutyric acid, α,β-diaminopropionic acid, homophenylalanine, and -tert-butylglycine.

The TCRs, polypeptides, and proteins of the invention (including functional variants thereof) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention (including functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3 rd ed., Cold Spring Harbor Press, Cold Spring Harbor, N.Y. 2001 ; and Ausubel et al., Current Protocols in Molecular Biology, Greene Publishing Associates and John Wiley & Sons, NY, 1994.

Further, some of the TCRs, polypeptides, and proteins of the invention (including functional variants thereof) can be isolated and/or purified from a source, such as a plant, a bacterium, an insect, a mammal, e.g., a rat, a human, etc. Methods of isolation and purification are well-known in the art. Alternatively, the TCRs, polypeptides, and/or proteins described herein (including functional variants thereof) can be commercially synthesized by companies, such as Synpep (Dublin, Calif.), Peptide Technologies Corp. (Gaithersburg, Md.), and Multiple Peptide Systems (San Diego, Calif.). In this respect, the inventive TCRs (including functional variants thereof), polypeptides, and proteins can be synthetic, recombinant, isolated, and/or purified. Included in the scope of the invention are conjugates, e.g., bioconjugates, comprising any of the inventive TCRs, polypeptides, or proteins (including any of the functional variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol. 298: 209-223 (2005) and Kirin et al, Inorg Chem. 44(15): 5405-5415 (2005)).

The term “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1 -methylinosine, 2,2-dimethylguanine, 2-methyladenine, 2-methylguanine, 3-methylcytosine, 5-methylcytosine, N-6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), butoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can comprise any nucleotide sequence which encodes any of the TCRs, polypeptides, proteins, or functional variants thereof described herein. For example, the nucleic acid can comprise, consist, or consist essentially of any one or more of the nucleotide sequence SEQ ID NOs: 21-29 or a combination thereof.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs (including functional portions and functional variants thereof). It is generally appreciated that conditions can be rendered more stringent by the addition of increasing amounts of formamide. The invention also provides a nucleic acid comprising a nucleotide sequence that is at least about 70% or more, e.g., about 80%, about 90%, about 91%o, about 92%, about 93%, about 94%, about 95%, about 96%, about 97%, about 98%, or about 99% identical to any of the nucleic acids described herein.

The nucleic acids of the invention can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λθTIO, λσΠI, ZapII (Stratagene), EMBL4, and λNMI 149, also can be used. Examples of plant expression vectors include pBIO1 , pBI101.2, pBI101.3, pBI121 and ρBΓN19 (Clontech). Examples of animal expression vectors include pEUK-Cl, pMAM and pMAMneo (Clontech). Preferably, the recombinant expression vector is a viral vector, e.g., a retroviral or a lentiviral vector.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2 μ plasmid, λ, SV40, bovine papilloma virus, and the like. Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter operably linked to the nucleotide sequence encoding the TCR, polypeptide, or protein (including functional variants thereof), or to the nucleotide sequence which is complementary to or which hybridizes to the nucleotide sequence encoding the TCR, polypeptide, or protein (including functional variants thereof). The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression. Further, the recombinant expression vectors can be made to include a suicide gene.

As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

Another embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DHSa E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5oc cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell.

For purposes herein, the T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. More preferably, the T cell is a T cell isolated from a human. The T cell can be any type of T cell and can be of any developmental stage, including but not limited to, CD4 +CD8+ double positive T cells, CD4+ helper T cells, e.g., Th1 and Th 2cells, CD8+ T cells (e.g., cytotoxic T cells), CD3+ T cells, Natural Killer T (NKT) cells, tumor infiltrating lymphocytes (TILs), memory T cells (e.g., central memory T cells and effector memory T cells), naive T cells, and the like.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The invention further provides an antibody, or antigen binding portion thereof, which specifically binds to a functional portion of any of the TCRs (or functional variant thereof) described herein. Preferably, the functional portion specifically binds to the cancer antigen, e.g., the functional portion comprising the amino acid sequence of CDR1 of a chain, of CDR2 of a chain, of CDR3 of a chain, of CDR1 of β chain, of CDR2 of β chain, of CDR3 of β chain, of variable region of a chain, of variable region of β chain or a combination thereof, e.g., In a preferred embodiment, the antibody, or antigen binding portion thereof, binds to an epitope which is formed by all 6 CDRs (CDRl-3 of the alpha chain and CDRl-3 of the beta chain). The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the functional portion of the inventive TCR (or functional variant thereof). Desirably, the antibody is specific for the functional portion of the inventive TCR (or functional variants thereof), such that there is minimal cross-reaction with other peptides or proteins.

Methods of testing antibodies for the ability to bind to any functional portion or functional variant of the inventive TCR are known in the art and include any antibody-antigen binding assay, such as, for example, radioimmunoassay (RIA), ELISA, Western blot, immunoprecipitation, and competitive inhibition assays (see, e.g., Janeway et al., infra, and U.S. Patent Application Publication No. 2002/0197266 A1).

Suitable methods of making antibodies are known in the art. For instance, standard hybridoma methods are described in, e.g., Kohler and Milstein, Eur. J. Immunol. , 5, 511-519 (1976), Harlow and Lane (eds.), Antibodies: A Laboratory Manual, CSH Press (1988), and C. A. Janeway et al. (eds.), Immunobiology, 5 thEd., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1.

Phage display furthermore can be used to generate the antibody of the invention. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al, supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in, for example, U.S. Pat. No. 5,639,641 and Pedersen et al., J. Mol. Biol., 235, 959-973 (1994). The invention also provides antigen binding portions of any of the antibodies described herein. The antigen binding portion can be any portion that has at least one antigen binding site, such as Fab, F(ab′) 2, dsFv, sFv, diabodies, and triabodies.

A single-chain variable region fragment (sFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)). Antibody fragments of the invention, however, are not limited to these exemplary types of antibody fragments.

Also, the antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

The inventive TCRs, polypeptides, proteins, (including functional variants thereof), nucleic acids, recombinant expression vectors, host cells (including populations thereof), and antibodies (including antigen binding portions thereof), can be isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70%, 80%, 90%, 95%, or can be 100%.

The inventive TCRs, polypeptides, proteins (including functional variants thereof), nucleic acids, recombinant expression vectors, host cells (including populations thereof), and antibodies (including antigen binding portions thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, functional portions, functional variants, nucleic acids, expression vectors, host cells (including populations thereof), and antibodies (including antigen binding portions thereof), and a pharmaceutically acceptable carrier. The inventive pharmaceutical compositions containing any of the inventive TCR materials can comprise more than one inventive TCR material, e.g., a polypeptide and a nucleic acid, or two or more different TCRs (including functional portions and functional variants thereof). Alternatively, the pharmaceutical composition can comprise an inventive TCR material in combination with another pharmaceutically active agents or drugs, such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive

TCR material under consideration. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for oral, aerosol, parenteral, subcutaneous, intravenous, intramuscular, intra-arterial, intrathecal, or intraperitoneal administration. More than one route can be used to administer the inventive TCR materials, and in certain instances, a particular route can provide a more immediate and more effective response than another route. Preferably, the inventive TCR material is administered by injection, e.g., intravenously. When the inventive TCR material is a host cell expressing the inventive TCR (or functional variant thereof), the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumin.

In an embodiment of the invention, the pharmaceutical composition may further comprise another therapeutic agent such as a chemotherapeutic agent.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a hematological antigen, or detect, treat or prevent hematological disorder in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated. Many assays for determining an administered dose are known in the art. For purposes of the invention, an assay, which comprises comparing the extent to which target cells are lysed or T cell cytokines (such as IFN-γ or IL-6 or Il-2) are secreted by T cells expressing the inventive TCR (or functional variant or functional portion thereof), polypeptide, or protein upon administration of a given dose of such T cells to a mammal among a set of mammals of which is each given a different dose of the T cells, could be used to determine a starting dose to be administered to a mammal. The extent to which target cells are lysed or IFN-γ, IL2 or IL-6 is secreted upon administration of a certain dose can be assayed by methods known in the art. The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the condition being treated. In an embodiment in which the inventive TCR material is a population of cells, the number of cells administered per infusion may vary, e.g., from about 1×10⁶ to about 1×10¹¹ cells or more.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a targeting moiety. The practice of conjugating compounds, e.g., inventive TCR materials, to targeting moieties is known in the art. See, for instance, Wadwa et al, J. Drug Targeting 3: 111 (1995) and U.S. Pat. No. 5,087,616. The term “targeting moiety” as used herein, refers to any molecule or agent that specifically recognizes and binds to a cell-surface receptor, such that the targeting moiety directs the delivery of the inventive TCR materials to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). The term “bridge” as used herein, refers to any agent or molecule that links the inventive TCR materials to the targeting moiety. One of ordinary skill in the art recognizes that sites on the inventive TCR materials, which are not necessary for the function of the inventive TCR materials, are ideal sites for attaching a bridge and/or a targeting moiety, provided that the bridge and/or targeting moiety, once attached to the inventive TCR materials, do(es) not interfere with the function of the inventive TCR materials, i.e., the ability to bind to antigen as parent TCR; or to improve exogenous alpha and beta TCR chain pairing while eliminating or minimizing unproductive mixed dimers (endogenous/transduced dimers), or to detect, treat, or prevent a hematological disorder.

It is contemplated that the inventive pharmaceutical compositions, TCRs (including functional variants thereof), polypeptides, proteins, nucleic acids, recombinant expression vectors, host cells, or populations of cells can be used in methods of treating or preventing a hematological disorder. Without being bound to a particular theory, the inventive TCRs (and functional variants thereof) are believed avoid mispairing, such that the TCR (or related inventive polypeptide or protein and functional variants thereof) when expressed by a cell is able to mediate an effective immune response against a target cell. In this regard, the invention provides a method of treating or preventing a hematological tumor, a solid tumor or an infective disease in a mammal, comprising administering to the mammal any of the pharmaceutical compositions, TCRs (and functional variants thereof), polypeptides, or proteins described herein, any nucleic acid or recombinant expression vector comprising a nucleotide sequence encoding any of the TCRs (and functional variants thereof), polypeptides, proteins described herein, or any host cell or population of cells comprising a recombinant vector which encodes any of the TCRs (and functional variants thereof), polypeptides, or proteins described herein, in an amount effective to treat or prevent an hematological disorder in the mammal.

In an embodiment of the invention, the inventive methods of treating or preventing hematological disorder may further comprise co-administering MHC Class I restricted TCRs, or polypeptides, proteins, nucleic acids, or recombinant expression vectors encoding MHC Class I restricted TCRs, or host cells or populations of cells expressing MHC Class I restricted TCRs, to the mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Also provided is a method of detecting the presence of a hematological tumor, a solid tumor or an infective disease in a mammal. The method comprises (i) contacting a sample comprising cells of the hematological tumor, solid tumor or an infective disease with any of the inventive TCRs (and functional variants thereof), polypeptides, proteins, nucleic acids, recombinant expression vectors, host cells, populations of cells, or antibodies, or antigen binding portions thereof, described herein, thereby forming a complex, and (ii) detecting the complex, wherein detection of the complex is indicative of the presence of the a hematological tumor, solid tumor or

infective disease in the mammal.

With respect to the inventive method of detecting a hematological tumor, a solid tumor or an infective disease in a mammal, the sample of cells of the cancer or infection can be a sample comprising whole cells, lysates thereof, or a fraction of the whole cell lysates, e.g., a nuclear or cytoplasmic fraction, a whole protein fraction, or a nucleic acid fraction.

For purposes of the inventive detecting method, the contacting can take place in vitro or in vivo with respect to the mammal. Preferably, the contacting is in vitro. Also, detection of the complex can occur through any number of ways known in the art. For instance, the inventive TCRs (and functional variants thereof), polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal. Still preferably, the cells are allogeneic to the mammal. The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

As used herein, the term “tumor” or “tumor disease” means both benign and malignant tumors or neoplasms and includes melanomas, myelomas, lymphomas, leukemias, carcinomas and sarcomas. Illustrative examples of tumors are cutaneous tumours such as malignant melanomas and mycosis fungoides; hematologic tumors such as leukemias, for example, acute lymphoblastic, acute myelocytic, or chronic myelocytic leukemia; lymphomas such as Hodgkin's disease or malignant lymphoma; gynecologic tumors such as ovarian and uterine tumors; urologic tumors such as those of the prostate, bladder, or testis; soft tissue sarcomas, osseous, or non-osseous sarcomas, breast tumors; tumors of the pituitary, thyroid, and adrenal cortex; gastrointestinal tumors such as those of the esophagus, stomach, intestine, and colon; pancreatic and hepatic tumors; laryngeae papillomatosis and lung tumors. Preferred tumors in the context of the present invention are selected from melanoma, multiple myeloma, lung tumor, endometrial tumors, glioma, lymphoma, leukemia or prostate tumor.

The provided compounds of the invention are in a further aspect for use in medicine, for example for use in the treatment of a cancerous disease. Most preferably the compounds of the invention are used in a cancer treatment that involves an adoptive T-cell transfer. Yet another aspect of the invention relates to a method of treating a human subject, specifically human subject suffering from a tumor disease. The method of treatment comprises the administration of any of the aforementioned compounds into a patient in need of such a treatment. The administration of the compounds of the invention can for example involve the infusion of T cells of the invention into said patient. Preferably such T cells are autologous T cells of the patient which were in vitro transduced with a nucleic acid or TCR of the present invention. Thus also provided is a pharmaceutical composition, comprising a TCR or TCR fragment according to the invention, or a nucleic acid, a vector, a host cell, or an isolated T cell according to the invention. In a preferred embodiment the pharmaceutical composition is for immune therapy.

Examples of pharmaceutically acceptable carriers or diluents useful in the present invention include stabilizers such as SPGA, carbohydrates (e.g. sorbitol, mannitol, starch, sucrose, glucose, dextran), proteins such as albumin or casein, protein containing agents such as bovine serum or skimmed milk and buffers (e.g. phosphate buffer). The exact formulation, administration route and dosage can be selected by the technician taking into account the status of the patient.

Optionally the TCR of the invention are derivatized with a pharmacokinetic (PK) modifiying agent such as a fatty acid, a homing peptide, a transport agent, a cell-penetrating agent, an organ-targeting agent, or a chelating agent.

Techniques for the formulation or preparation and application/medication of the active components of the present invention are published in “Remington's Pharmaceutical Serives”, Mack Publishing Co., Easton, Pa., latest edition”. The dosing regimen can be done as a single dose or multiple doses at intervals. Dose amounts and time intervals can be adjusted individually to provide the therapeutic effect giving an improvement of the symptoms or a prolongation of patient survival.

To obtain a cell expressing the TCR of the invention, any vector or suitable method (like for example transduction of a nucleic acid or vector, transfection of RNA, transposon, electroporation) which allows the expression of TCR exogenous in a T cell can be used. The methods of transduction to introduce nucleic acid molecules in T cells are well known in the art and include for example vehicles of viral transduction.

The cells of the invention can be administered to a patient in order to express the TCR of interest (Adoptive Therapy).

Abbreviations used herein are: amu or amu: alpha mutated; awt or αwt: alpha wild type; amubmu: alpha mutated/beta mutated; amubwt: alpha mutated/beta wild type; awtbwt: alpha wild type/beta wild type; awtbmu: alpha wild type/beta mutated; bmu or βmu: beta mutated; bwt or βwt: beta wild type; BSA: Bovine Serum Albumin; DMEM: Dulbecco's Modified Eagle Medium; EBV: Epstein-Barr Virus; FACS: Fluorescence Advanced Cell Sorting; FCS: Fetal Calf Serum; FLIM: Fluorescence-Lifetime Imaging Microscopy; FRET: Fluorescence Resonance Energy Transfer; IFNγ: Interferon γ; IL-2: Interleukin-2; MHC: Major Histocompatibility Complex; pMSGV: Murine Stem Cell Virus (MSCV)-based splice-gag vector; MTX: Methotrexate; PBS: Phosphate Buffered Saline; RPMI: Roswell Park Memorial Institute medium; SPGA: Sucrose-Phosphate-Glutamate-Albumin; TCR: T-cell receptor; TM: Trans-Membrane; TSA: Tumor Specific Antigen.

The present invention will be described by non-limitative examples with reference to the following figures:

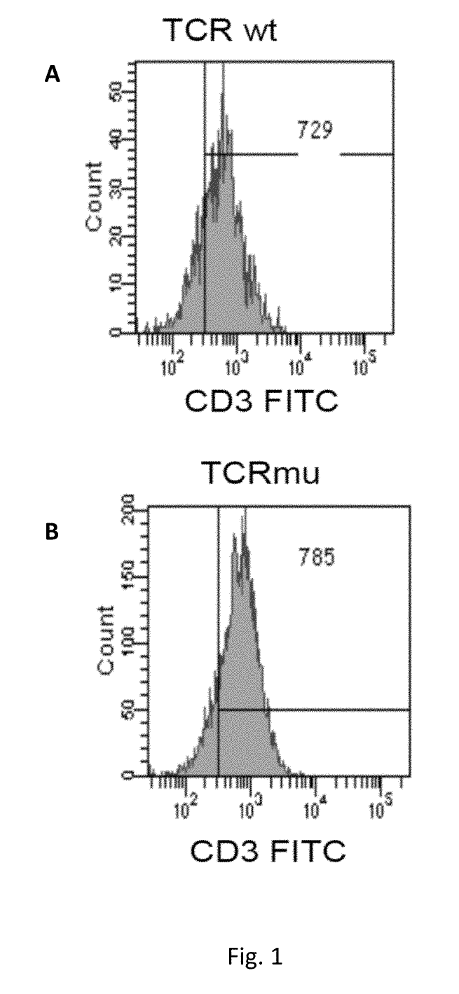

FIG. 1. Membrane CD3 expression of 54ζ17 cells obtainable as described in (8) after transfection (A) of αwt (SEQ ID NO:1) and βwt (SEQ ID NO:3) chains or (B) αmu (SEQ ID NO:2) and βmu (SEQ ID NO:4) chains. The transfected chains correspond to the TCR specific for HIV Reverse Transcriptase (HIV-RT) 248-262 peptide. The figure represents a cytofluorimetric analysis of the fluorescence intensity of the 54ζ17 cells transfected and labeled with antibody anti-CD3ε conjugated to FITC 145-2c11 (eBioscience cat 11-0031-82). Expression of the correctly associated receptor induces membrane localization of the CD3ε chain in hybridoma cells 54ζ17. Numbers represents the mean of fluorescence intensity values.

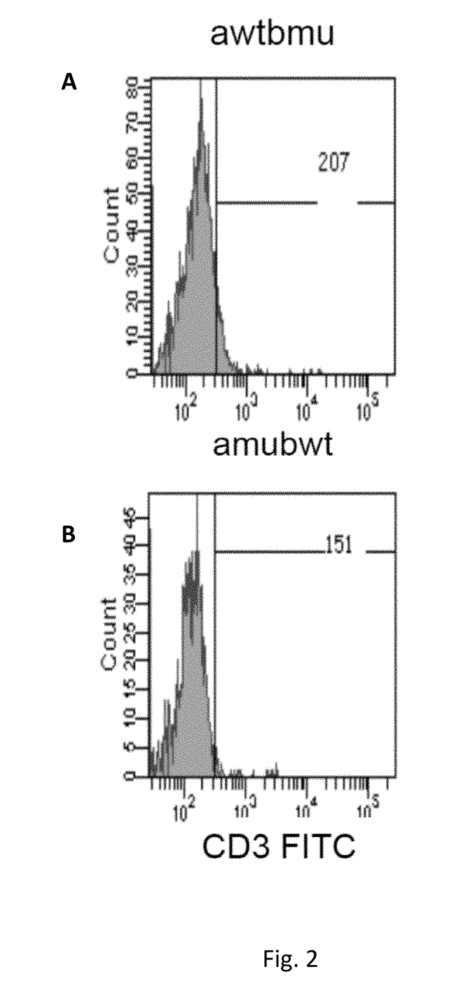

FIG. 2. Membrane CD3 expression of 54ζ17 cells after transfection of awt (SEQ ID NO:1)/βmu (SEQ ID NO:4) (A), or αmu (SEQ ID NO:2)βwt (SEQ ID NO:3) (B). Mispairing of the two TCR chains does not induce membrane localization of CD3ε on T cell surface nor the staining of 54ζ17 cells with the specific antibody. Numbers represents the mean of fluorescence intensity values.

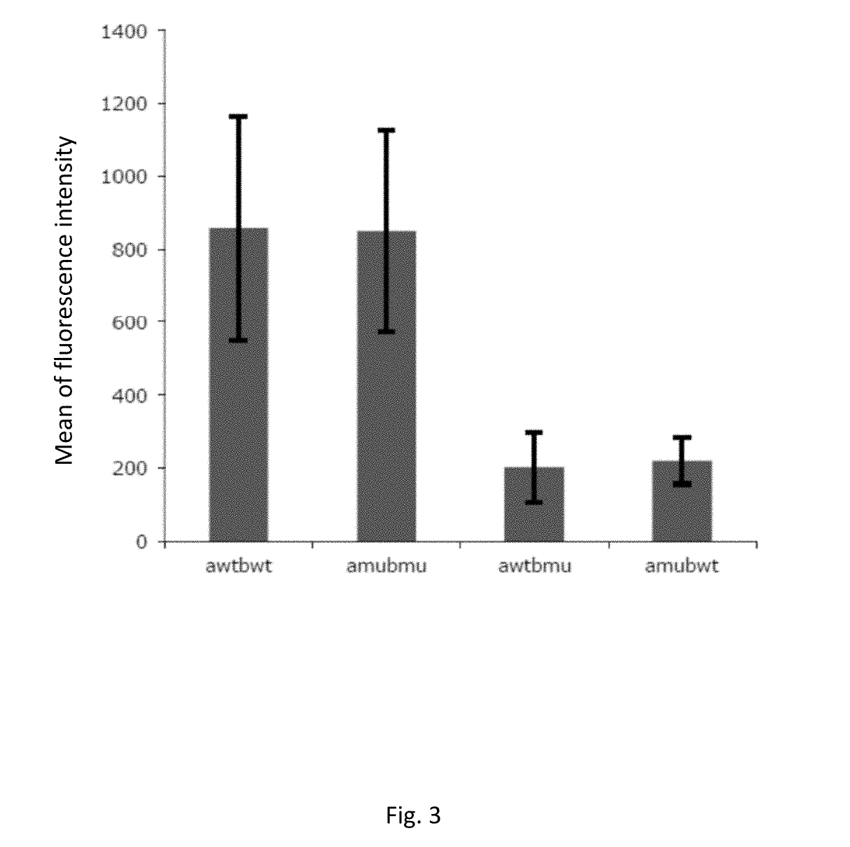

FIG. 3. Mean of fluorescence intensity of CD3 expression of 54ζ17 cells transfected with the different TCRα and β combinations (mean of 3 experiments). awtbwt: SEQ ID NO:1/SEQ ID NO:3; amubmu: SEQ ID NO:2/SEQ ID NO:4; awtbmu: SEQ ID NO:1/SEQ ID NO:4; amubwt: SEQ ID NO:2/SEQ ID NO:3. Mean fluorescence intensity is reported on the Y-axis.

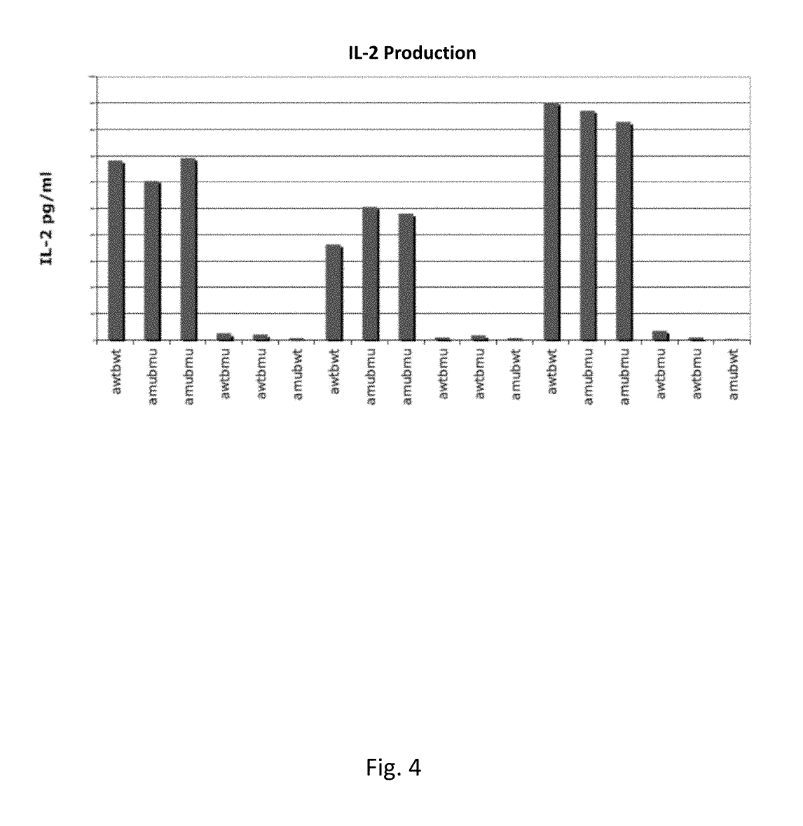

FIG. 4. IL-2 production for several independent T cell 54ζ17 hybridomas transfected with awt (SEQ ID NO:1) bwt (SEQ ID NO:3) TCR, amu (SEQ ID NO:2) bmu (SEQ ID NO:4) TCR, awt (SEQ ID NO:1) bmu (SEQ ID NO:4) TCR or amu (SEQ ID NO:2) bwt (SEQ ID NO:3) TCR, when stimulated with specific antigen HIV reverse transcriptase (RT) (248-262) peptide. IL-2 secreted was measured by ELISA (Biolegend, see Materials and Methods). The histogram shows how the 54z17 hybridoma reconstituted with the amu bmu chains is able to transduce the signal after specific TCR engagement and lead to the production of IL-2 to the same extent as the 54ζ17 reconstituted with the awt bwt TCR. The 54ζ17 hybridomas reconstituted with awt bmu chains or amu bwt (chains produce a significant smaller amount of IL-2.

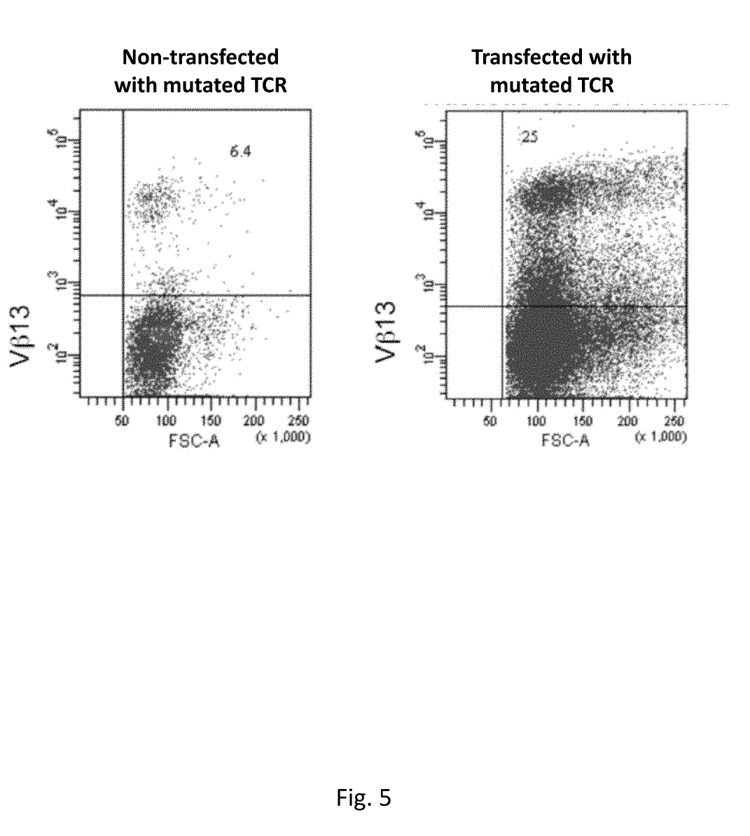

FIG. 5. Cytofluorimetric analysis showing the expression of the mutated TCR through retroviral infection using a monoclonal antibody anti-Vβ13 PercP-coniugated clone MP12-3 eBioscience cat 46-5797-80 (specific for the transfected beta chain). The figure shows TCR expression (visualized through antibody anti beta chain (anti-Vβ13 PercP-coniugated clone MP12-3 eBioscience cat 46-5797-80) in murine splenocytes non-transfected or transfected with a retrovirus encoding the mutated alpha (SEQ ID NO:2) and mutated beta (SEQ ID NO:4) chains object of the present invention. The anti-Vβ13 positivity seen in the non-transfected sample reflects the background value of said chain expression in the set of a wild type mouse. Numbers represent the percentage of cells labeled by the specific antibody. FSC-A: Forward Scatter-Area.

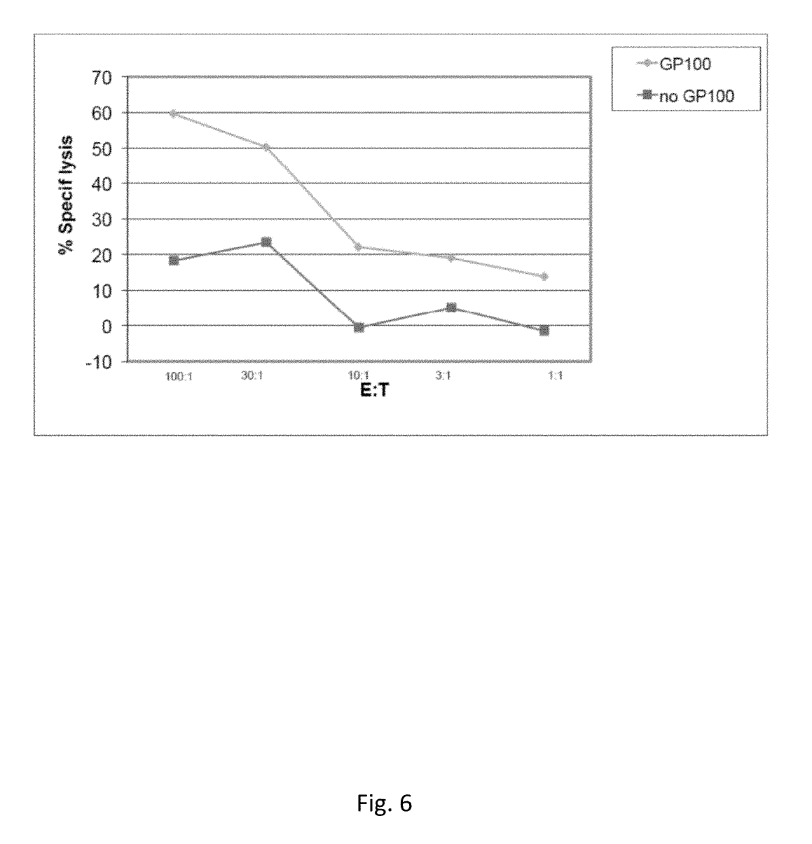

FIG. 6. Cytotoxic activity of murine T cells (Effector, E) transfected with a retroviral vector encoding the TCR specific for the gp100 antigen and comprising the transmembrane modifications object of the present invention (alpha chain: SEQ ID NO: 2; beta chain: SEQ ID NO: 4). EL4 cells (ATCC® Number: TIB-39™, (11)) were used as target cells (Target, T), conjugated with calcein and pre-incubated with the antigenic gp100 peptide. The light gray graph represents the response against target cells pre-incubated with the antigenic gp100 peptide while the dark gray graph shows the control cytotoxicity against target cells not pre-incubated with the gp100 peptide.

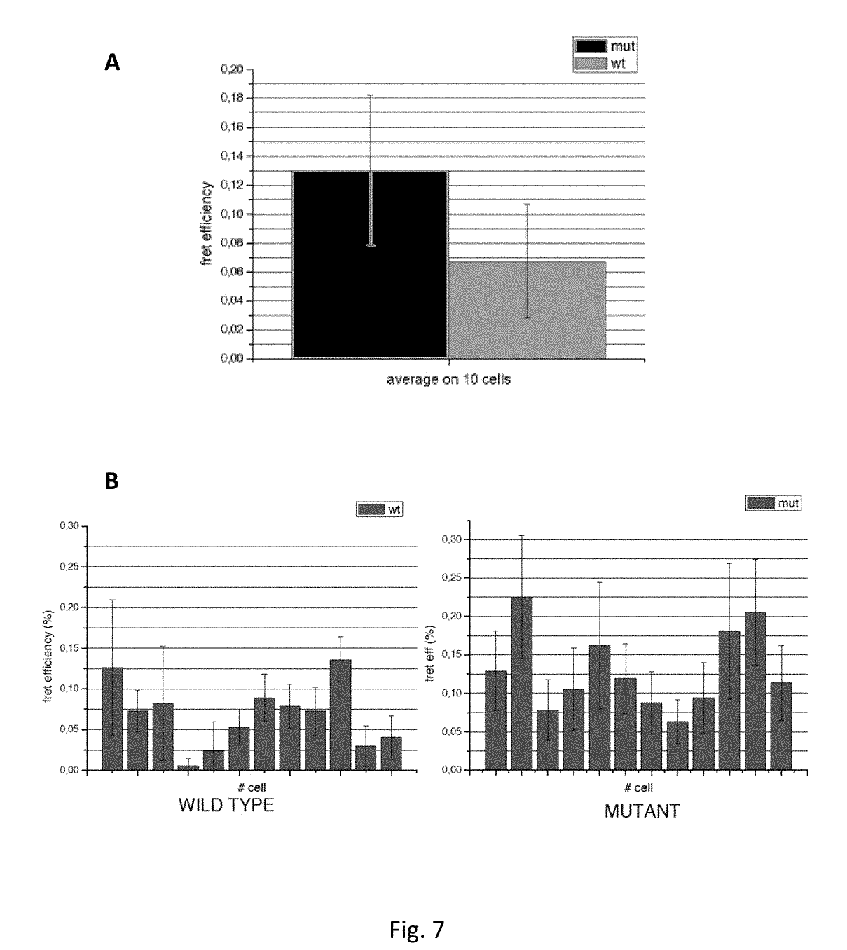

FIG. 7. FRET analysis on mouse splenocytes infected with TCR OT-I (Charles River C57BL/6-Tg (TcraTcrb)1100Mjb/CrlC) which recognizes the OVA₂₅₇₋₂₆₄ (SIINKFEL) peptide. Splenocytes were transduced with wild type or with the TCR comprising the transmembrane modification of SEQ ID:2 and SEQ ID:4. A) Mean +/−SD of FRET signals on splenocytes infected with TCR wt or TCR mutated is shown (average of 10 cells) B) FRET efficiency values are reported at single cell level.

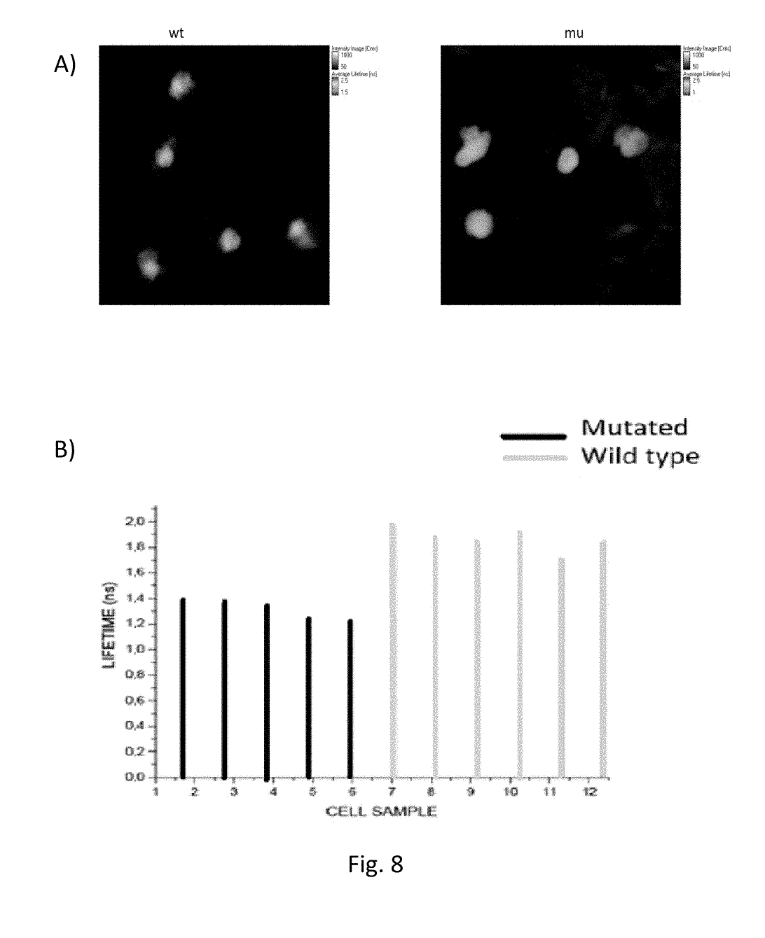

FIG. 8. FLIM (Fluorescence Lifetime Imaging Microscopy) analysis illustrating the half-life of fluorescence signals following interactions of Vα 2 and Vβ 5 antibody labelled with a donor/acceptor dye pair. Cells were analyzed at confocal microscope using FLIM and the software package SymphoTime (PicoQuant) was used to process FRET data. A) Donor lifetime revealed pixel by pixel: FRET is expressed as color scale. B) Differences in FRET efficiencies between the mutant and wild type are reported in the histogram. The lifetime of the donor will decrease in the presence of the acceptor. The lifetime (nanoseconds) fitted to the distribution model is reported on Y-axis.

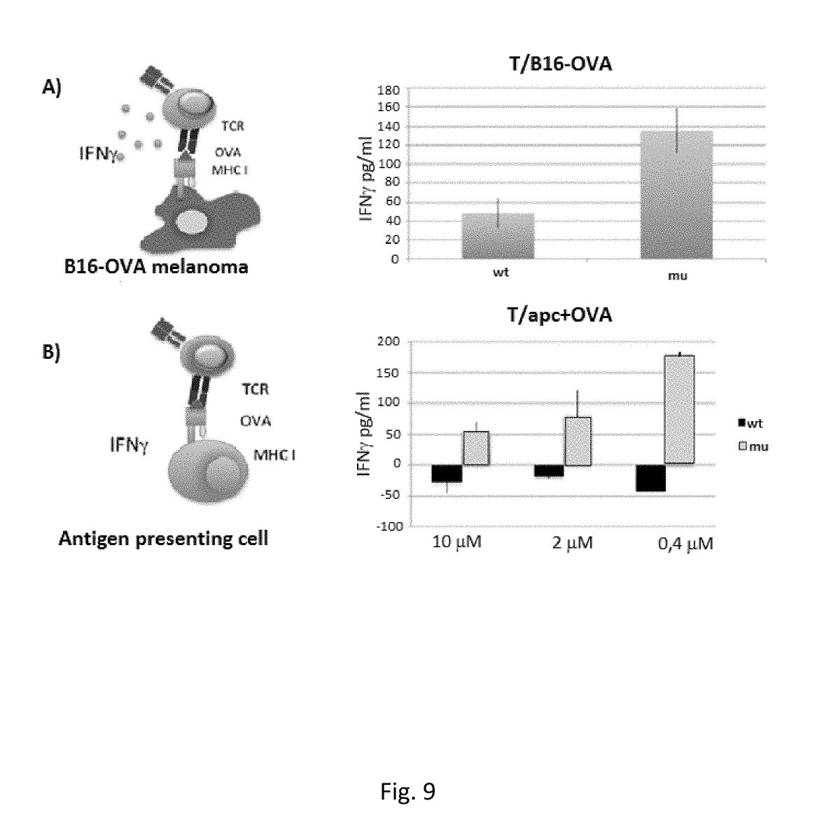

FIG. 9. Functional activity of CD8+ T lymphocytes isolated from the spleen and transduced with wild type or mutated TCR OT-I which specifically recognizes the OVA₂₅₇₋₂₆₄ peptide SIINFEKL. Production of IFNγ (A) by CD8 T cells co-cultured with B16 melanoma cells expressing the ovalbumin protein (T/B16-OVA) and (B) by CD8 T cells co-cultured with antigen presenting cells pre-pulsed with OVA₂₅₇₋₂₆₄ peptide (T/apc+OVA) at the concentrations of 10 μM, 2 μM and 0.4 μM. The IFNγ concentrations are calculated by interpolation of the experimental optical density values on a standard calibration curve. The standard calibration curve is obtained correlating known IFNγ concentrations and optical density of the colorimetric assay. Negative concentration values are obtained if the experimental optical density is lower than the calibrator (negative value means zero).

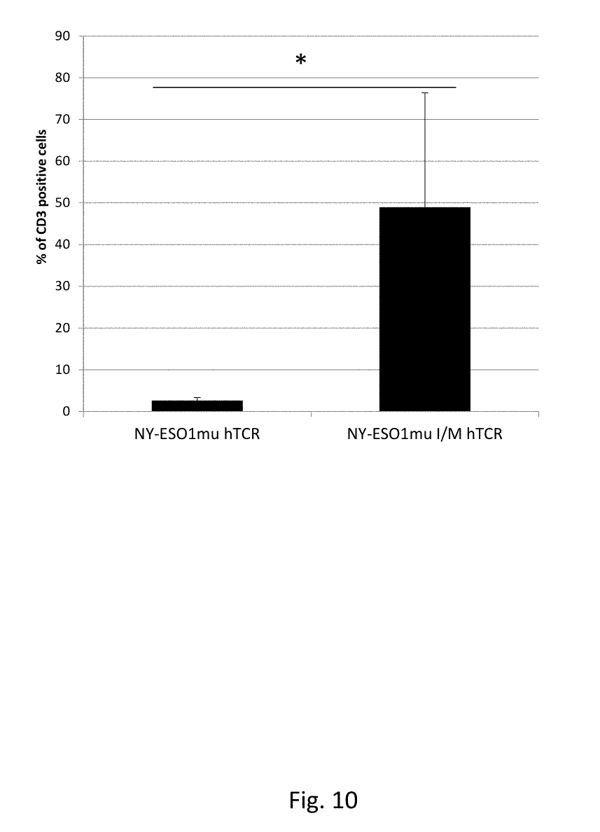

FIG. 10. Expression in Jurkat J76 cell line (TCR defective). The percentage of CD3 positive cells measured 24 hours after the infection with retrovirus encoding the mutant TCR (comprising mutated alpha chain: SEQ ID. NO: 15 and mutated beta chain: SEQ ID NO:18) specific for the human Ny-Esol antigen (left bar) and the mutant plus I/M TCR (comprising mutated alpha chain: SEQ ID. NO: 16 and mutated beta chain: SEQ ID NO:18) specific for the human Ny-Esol antigen (right bar) is reported. The experiment has been performed four times. *=p<0.05.

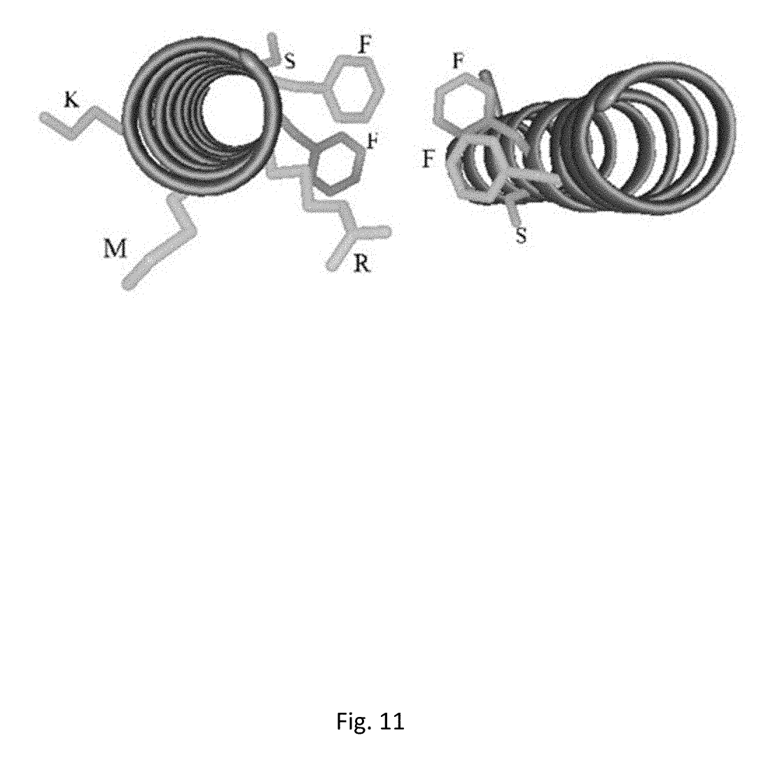

FIG. 11. Molecular modeling of the transmembrane region of mutated human TCR. The aminoacids of the FXXXFXXS (aa 1 to 8 of SEQ ID No: 30) motif are reported. The Lysine aminoacid responsible for the interaction with the epsilon-delta CD3 dimer, the Arginin interacting with the zeta-zeta dimer and the Methionine introduced instead of the Isoleucin to induce a better TCR expression.

### DETAILED DESCRIPTION OF THE INVENTION

Materials and Methods

Hybrid TCRs Generation and Cloning

Cloning of TCR alpha and beta chains was performed as previously described [8]. The mutated TCR alpha and beta chains have been obtained by introducing the mutation by overlap extension PCR using the primers listed below.

1 mCcSSTCRF:
(SEQ ID NO: 5)
5′GGATCCAATATTCAGAACCCAGAACCTGCTGTG 3′
2 CaTCRmuR:
(SEQ ID NO: 6)
5′CAGCGTCATGAGCAGGCTAAATCCAAATACTTTCAGAAAGAGGATTC
GG 3′
3 CaTCRmuF:
(SEQ ID NO: 7)
5′CCGAATCCTCTTTCTGAAAGTATTTGGATTTAGCCTGCTCATGACGC
TG 3′
4 mCaNotITCRR:
(SEQ ID NO: 8)
5′CTGCAGGCGGCCGCGTGAGGAGGACGGAC 3′
5 mCbeta Eco0109 f:
(SEQ ID NO: 9)
5′ TCTAGAGGACCTGAGAAATGTG 3′
6 mCbetaXba:
(SEQ ID NO: 10)
5′GGTCGACTCTAGAACTAGTGGATCC 3′
7 tcrb mutf:
(SEQ ID NO: 11)
5′ ATCCTATTCGGGAAGGAAGGCCTTCCTATATTCT 3′
8 tcr mu r:
(SEQ ID NO: 12)
5′ GCACAGAATATAGGAAGGCCTTCCCGAATAGGAT 3′

Primers 1 2, 3 and 4 were used for alpha chain amplification and primers 5,6,7 and 8 for beta chain amplification.

Experiments on hybridoma cells have been performed as previously described [8].

Human TCR alpha and beta chains were obtained by GeneART® gene synthesis service (Termo Fisher Inc.).

The transfection of isolated T cells from murine splenocytes was done as previously described by using retroviral vectors encoding a T receptor specific for the pMel melanoma antigen [9]. The starting vector is described in [12] and was mutated as above described.

Cells

The murine T hybridoma α⁻β⁻ transfected with human CD4 and murine ζ chain (T54ζ17) has been previously described [8]. Cells were maintained in DMEM supplemented with 10% FCS, 25U/mL penicillin G, 25 μg/mL streptomycin and 0.05 μM β-Mercaptoethanol. The 293T cell line, originally referred as 293tsA1609neo (Lenti-X™ 293T Cell Line 632180 clontech) is a highly transfectable derivative of human embryonic kidney 293 cells, and contains the SV40 T-antigen. Cells were cultured in DMEM supplemented with 10% FCS, 25 U/mL penicillin G and 25 ug/mL streptomycin.

Murine lymphocytes were cultured in RPMI 1640 supplemented with 10% fetal bovine serum, 2 mmol/L L-glutamine, 50 U/mL penicillin, 50 mg/mL streptomycin, 0.05 μM β-Mercaptoethanol, 1 mM sodium pyruvate and 20 IU/mL rhIL-2. Murine RMA-S is an antigen processing-defective cell line, obtained from a Rauscher virus-induced tumor [25]. The cells express only a low level of cell surface major histocompatibility complex (MHC) class I molecules, which are supposed to be devoid of internally derived antigenic peptides. Cells were maintained in RPMI 1640 supplemented with 10% FCS, 25U/mL penicillin G and 25 μg/mL streptomycin.

EBV cells (L-cell lines used as Antigen presenting cells produced by Epstein Barr virus infection of B cell from a healty donor as MHC matching Antigen presenting cells) were maintained in DMEM supplemented with 10% FCS, 25 U/ml penicillin G, 25 μg/ml streptomy cin.

Construction of Retroviral Vectors

The sequence encoding for the wild type and mutated alpha and beta chains of the invention (OVA specific TCR, gp100 specific TCR, and NY-ESO1 specific TCR) were produced by classical PCR reaction or by gene synthesis and introduced by digestion using the BglII-BamHI restriction enzymes in pMSGV backbone [26].

γ-Retrovirus Production

γ-Retrovirus was obtained by transfecting Human Embrionic Kidney (HEK) 293T, which stably express the SV40 Large T antigen with Lipofectamine 2000 reagent in DMEM medium (Gibco).

One day prior transfection HEK293T cells were seeded at 3.5×10⁶ cells into a 10 cm plate with 10 mL of complete medium and incubated at 37° C., 5% CO₂.

9 ug of plasmid coding for wild type or mutated TCRs of the invention (MSGV-OVA TCR wild type or mutated, MSGV-gp100 TCR wild type or mutated, MSGV-NY-ESO1 TCR wild type or mutated), 6 ug of plasmid encoding retroviral packaging system (pECOTROPIC Vector), 3 ug of plasmid coding for the protein VSVG to mediate viral entry through lipid binding and plasma membrane fusion and 30 uL of lipofectamine 2000 were used for co-transfection. 4-6 hours after transfection cell culture are supplemented with fresh complete growth medium and incubated at 37° C., 5% CO₂.

48 hours after the cell culture supernatant is collected, filtrated using 0.45 μm polysulphonate filter to remove cellular debris and concentrated by ultracentrifugation in SW28 rotor at 16500 rpm 1 h 30 min 4° C. Retroviral pellet is suspended in PBS and used for cell infection or stored at −80° C.

Transfection of Hybridoma Cell Line.

T 54ζ17 (58α⁻β⁻ previously transfected with human CD4 and the murine ζ chain) were suspended at 2×10⁷ cells/ml in DMEM 20% FCS. About 20 μg of expression vector encoding the wild type or mutated TCR b chain and 100 μg of expression vector were encoding the TCR a chain were added to 1 ×10⁷ cells/ml in a Gene Plus cuvette (Biorad, Richmond, Calif.) and electroporated at 250 V, 500 pf. After 48 h of incubation at 37° C., the cells were plated at 2×10⁴ cells/well in flat-bottom 96-well plates and placed in selective medium containing 400 nM MTX (Sigma) and 1.65 μM puromycin (Sigma).

Transduction of Primary Murine T Cells.

Mice were sacrificed, spleen were explanted and disaggregated. Red cells were lysed using 1 mL of Red blood cell Lysing buffer (Sigma) and incubated 5 min at RT. Cells were washed and suspended at 1×10⁶cells/mL. Splenocytes were activated using 10 ug/mL of anti-mouse CD32c11 antibody coated on plate and 4 ug/mL of anti-mouse CD28 antibody (Miltenyi Biotec Inc.). After 6 hours 20 units/well of human recombinant IL-2 were added to the culture. 2 days after activation CD8+ T cells were isolated and infected with 20 ul of retrovirus encoding mutated or wild type TCR. Cells were spinoculated 99 min a 2000 rpm a RT and 100 units/well of human recombinant IL-2 were added to the culture. Percentage of transduced cells was evaluated through FACS staining 48 hours after transduction.

Transduction of Jurkat 76 T Cells.

To infect the Jurkat J76 (TCR deficient cell line [24]) with retrovirus encoding wild type or mutated TCRs specific for NY ESO1 antigen, 48 hours after the transfection retroviral supernatant has been collected and filtered by using 0,45micron filter. 10m1 of filtered viral supernatant have been used to culture 500.000 Jurkat cells. The day after the infection cells have been spinned down and resuspended in 4 mL of RPMI 10% FCS medium (FIG. 11).

Flow Cytometry

To evaluate the expression of the TCR specific for the HIV reverse transcriptase (RT) (248-262) peptide on hybridoma cells T54ζ17, FITC-conjugated monoclonal antibody against murine CD3ε chain was used. Results are presented in FIG. 1 and FIG. 2. OVA₂₅₇₋₂₆₄ (SIINKFEL, (SEQ ID No: 32))-TCR transduced CD8+T cells were stained with FITC-conjugated monoclonal antibody against the variable region of the specific murine α chain Vα2 (eBioscience, San Diego, Calif.) and APC-conjugated monoclonal antibody against the variable region of the specific murine β chain Vβ5 (eBioscience, San Diego, Calif.). PerC-P conjugated anti-mouse Vb13.1 (Beckman Coulter, Miami, Fla.) was used to detect CD8+T cells transduced with TCR specific for KVPRNQDWL peptide (gp 100₂₅₋₃₃) (results are reported in FIG. 5). All the staining were performed according to manufacturer instructions. Cells were analyzed using a FACS CANTO flow cytometer and FACSDIVA software.

FRET (Fluorescence Resonance Energy Transfer)

For FRET acceptor photo bleaching, we used monoclonal antibody (Va2 BMS14-5812-82 eBioscience) specific for the murine variable region Vα2 conjugated with ALEXAfluor 568, as donor, and monoclonal antibody (139502 Biolegend) specific for the murine variable region Vβ5conjugated with ALEXAfluor 647, as acceptor. To perform FRET on primary murine T cells, such cells were seeded on coverslips. Coverslips were treated 20 min with 100 ul 1× poly-L-Lysine opportunely diluted with sterile PBS, and washed with 2 mL of PBS and air dry for 10 min in order to favor the adhesion. Primary murine T cells, activated as described above, were plated at 3×10⁶ cells/mL in complete medium on treated coverslip and incubated over night at 37° C., 5% CO₂. The day after, medium was removed and cells were fixed by incubation for 10 min at RT in 4% paraformaldehyde, and added of 1× Hoechst dye (Sigma) (fluorescent stain that specific binds to A-T rich DNA regions). Cells were then washed 5 min with PBS and incubated 20 min at RT with blocking solution (1× PBS, 0.5% BSA, 50 mM NH₄Cl). Subsequently cells were incubated 2 hours at RT with monoclonal antibody specific for the murine variable region Vα2 and monoclonal antibody specific for the murine variable region Vβ5, diluted 1:10 in blocking solution. Cells were washed with sterile water, coverslips were air dried and sealed with mowiol mounting solution. Cells were analyzed by using confocal microscopy by using P-FRET software package (ImageJ Plugins) to process FRET data.

FLIM: Time-Resolved FRET Analysis

To perform FLIM on primary murine T cells, such cells were treated as in FRET. Splenocytes transduced with wild type or mutated TCR and stained with monoclonal antibody specific for the murine variable region Vα2 conjugated with CF568, as donor, and monoclonal antibody specific for the murine variable region Vβ5 conjugated with CF647, as acceptor were analyzed at confocal microscope using FLIM (Fluorescence Lifetime Imaging Microscopy) and the software package SymphoTime (PicoQuant) was used to process FRET data.

Cytokine Release Assay

The ability of T54ζ17 hybridoma cells to produce IL-2, when stimulated with the specific antigen HIV reverse transcriptase (RT) (248-262) peptide, was tested in cytokine release assays (FIG. 4). In these assay 2×10⁴ hybridoma cells were co-cultured with 1×10⁵EBV cells pre-incubated overnight with 1 ug/mL or 0.1 ug/mL of synthetic antigen, in RPMI medium in a final volume of 0.2 mL in duplicate wells of a 96-well flat-bottom microplate. Culture supernatants were harvested 18-24 hours after the initiation of co-culture and assayed for IL-2 by ELISA (Biolegend).

TCR-engineered CD8+ T cells were tested for antigen-specific reactivity in cytokine release assays using antigen presenting cells (irradiated syngenic splenocytes) loaded with synthetic peptide or tumor cells expressing the antigen. In these assays, effector cells (1×10⁵) were co-cultured with equal number of target cells in RPMI complete growth medium in a final volume of 0.2 mL in duplicate wells of a 96-well flat-bottom microplate. Culture supernatants were harvested 18-24 hours after the initiation of co-culture and assayed for IFN-g by ELISA (Biolegend).

Cytotoxicity (Calcein Release Assay)

Effector cell-mediated cytotoxic activity was evaluated by the standard Calcein-AM release cytotoxicity assay (FIG. 6). Briefly, target cell lines were pulsed for 2 hours with synthetic antigen gp100 (Kawakami Y et al J. Immunol., 154:3961-3968, 1995; Primm Srl, Milan, Italy) and labeled with 15 μM Calcein-AM (C1359 Sigma) for 30 min at 37° C. and washed twice. Calcein AM is a non-fluorescent, hydrophobic compound that easily permeates intact, live cells. The hydrolysis of Calcein AM by intracellular esterases produces Calcein, a hydrophilic, strongly fluorescent compound that is well-retained in the cell cytoplasm. After cell lysis Calcein is released in the cell culture supernatant and can be detected) for 30 min at 37° C. and washed twice. Effector cells were incubated with 5×10³ target cells, at the indicated E:T ratios, for 5 h at 37° C. in 200 μl of complete medium. After short centrifugation, 75 μl of cell culture supernatant was harvested and transferred in a new plate and the Calcein release was measured by fluorescent plate reader at 490 nm excitation. The percentage of specific cytotoxicity was calculated as 100×(cpm experimental release−cpm spontaneous release)/(cpm maximum release−cpm spontaneous release). Maximum release was obtained by target cells cultured in medium plus 2% Triton X100. Spontaneous release was always ≤20%. All experimental points were performed in triplicate.

Analysis of Signal Transduction in Jurkat T Cells

To evaluate ERK phosphorylation, J76 cells infected with retrovirus encoding for TCR-Ny-Eso wild type or TCR-Ny-Eso mutated in TM region were stimulated with 1:25 diluted Ny-Eso tetramer conjugated to streptavidin-PE (Life technologies S866). Briefly 1×10⁵ cells/for time point were stimulated with tetramer at 37° C. with 1000 rpm shaking for 0″, 60″, 120″, 180″, 300″, 600″ and fixed with Phosflow Fix buffer (BD Phosflow Fix Buffer 1 cat.557870, BD

Bioscience). Afterwards cells were permeabilized with Phosflow Perm Buffer (BD PhosflowFix Perm II cat.558052, BD Bioscience) and stained for 1 hour at 37° C. in the dark with antiP-Erk Alexa-Fluor 647 conjugated antibody (Phospho-p44/42 MAPK (Erk1/2) clone E10 cat.4375, Cell Signaling Technology). Cells were washed and analyzed by using a FACS CANTO 2 flow cytometer.

### EXAMPLES

Description of Wild Type and Mutant TCR Chains

Genetically modified TCRs whose transmembrane regions contain three or four substituted amino acid residues in the alpha chain and three in the beta chain were obtained as described in methods. Below, the mutated amino acid residues are highlighted in bold and shown in the amino acidic sequence context of mutated and wild type transmembrane regions of the TCR components (TM, Trans Membrane):

Mus musculus:

| | |
|---|---|
| VMGLRILLLKVAGFNLLMTLRLW | wild type TM alpha (SEQ ID NO: 1) |
| VMGLRILFLKVFGFSLLMTLRLW | mutated TM alpha (SEQ ID NO: 2) |
| TILYEILLGKATLYAVLVSTLVV | wild type TM beta (SEQ ID NO: 3) |
| TILYEILFGKAFLYSVLVSTLVV | mutated TM beta (SEQ ID NO: 4) |
| TILYEILLGKATLYAVLVSGLVL | wild type TM beta (SEQ ID NO: 19) |
| TILYEILFGKAFLYSVLVSGLVL | mutated TM beta (SEQ ID NO: 20) |

Homo Sapiens:

| | |
|---|---|
| VIGFRILLLKVAGFNLLMTLRL | wild type TM alpha (SEQ ID NO: 14) |
| VIGFRILFLKVFGFSLLMTLRL | mutated TM alpha (SEQ ID NO: 15) |
| VMGFRILFLKVFGFSLLMTLRL | mutated TM alpha (SEQ ID NO: 16) |
| TILYEILLGKATLYAVLVSALVL | wild type TM beta (SEQ ID NO: 17) |
| TILYEILFGKAFLYSVLVSALVL | mutated TM beta (SEQ ID NO: 18) |

Mus Musculus
wild type TM alpha (SEQ ID NO: 1), nucleotide sequence (SEQ ID NO: 21)
gtgatgggcctgagaatcctgctgctgaaggtggccggcttcaacctgctgatgaccct
gaggctgtgg
mutated TM alpha (SEQ ID NO: 2), nucleotide sequence (SEQ ID NO: 22)
gtgatgggcctgagaatcctgTtCctgaaggtgTTcggcttcaGcctgctgatgaccct
gaggctgtgg
wild type TM beta (SEQ ID NO: 3), nucleotide sequence (SEQ ID NO: 23)
Accatcctgtacgagatcctgctgggcaaggccacactgtacgccgtgctggtgtccgg
cctggtgctg
mutated TM beta (SEQ ID NO: 4), nucleotide sequence (SEQ ID NO: 24)
accatcctgtacgagatcctgTTCggcaaggccTTCctgtacTCTgtgctggtgtccgg
cctggtgctg
Homo Sapiens:
wild type TM alpha (SEQ ID NO: 14), nucleotide sequence (SEQ ID NO: 25)
gtgattgggttccgaatcctcctcctgaaagtggccgggtttaatctgctcatgacgct
gcggctg
mutated TM alpha (SEQ ID NO: 15), nucleotide sequence (SEQ ID NO: 26)
gtgattgggttccgaatcctcTTTctgaaagtgTTTgggtttTCCctgctcatgacgct
gcggctg
mutated TM alpha (SEQ ID NO: 16), nucleotide sequence (SEQ ID NO: 27)
gtgATGgggttccgaatcctcTTTctgaaagtgTTTgggtttTCCctgctcatgacgct
gcggctg
wild type TM beta (SEQ ID NO: 17), nucleotide sequence (SEQ ID NO: 28)
accatcctctatgagatcttgctagggaaggccaccttgtatgccgtgctggtcagtgc
cctcgtgctg
mutated type TM beta (SEQ ID NO: 18), nucleotide sequence (SEQ ID NO: 29)
accatcctctatgagatcttgTTTgggaaggccTTTttgtatTCCgtgctggtcagtgc
cctcgtgctg

Since the transmembrane regions of alpha and beta TCR chains have conserved motifs (13) the mutations described above may be inserted in any pair of TCR chains.

Transfections in a Hybridoma Cell Line Deprived of Endogenous TCR

As a proof of principle, the above mutations were introduced into the TCR alpha and beta chain specific for the HIV reverse transcriptase (RT) (248-262) peptide that has been previously described by the inventors' laboratory [8] and has been reconstituted into the T cell hybridoma 54ζ17 (Blank U, Boitel B, Mege D, Ermonval M, Acuto O (1993) Eur J. Immunol 23:3057). Experiments are illustrated in FIGS. 1-4.

The TCR complex is assembled in the endoplasmic reticulum in a series of defined steps, in which the early and correct association of the TCRα and TCRβ chains is crucial. Correctly paired complexes are then exported to the surface of the cell in association with the CD3 γ, δ, ε and ζ chains.

The 54ζ17 T cell hybridoma is devoid of endogenous TCRα and β coding genes, and does not express CD3 proteins on the cell surface. Upon transfection of TCR α and β chain coding plasmids, TCR chains associate and induce CD3 expression on T cell surface.

The described TCR chains mutated into the transmembrane region are correctly associated after transfection in 54ζ17 T cell hybridoma, as demonstrated by antiCD3E antibody staining of transfected cell surface and cytofluorimetric analysis (FIG. 1).

Furthermore, as seen in FIGS. 2 and 3, mutated chains cannot associate with endogenous wild type chains, then mixed dimers are not formed. Indeed transfection of wild-type alpha chain and mutated beta chain or transfection of mutated alpha chain and wt beta chain generate TCRs that do not express the CD3ε chain on T cell surface (FIGS. 2, 3).

Transfection-Mediated IL-2 Production

The mutant TCR chains do not interfere with the correct signal transduction after TCR engagement, as demonstrated by ELISA for the IL-2 production after T cell activation by RT 248-262 peptide (FIG. 4).

Transduction of Isolated T-Cells from Murine Splenocytes

T cells isolated from murine splenocytes were transduced by using retroviral vectors properly engineered and encoding a TCR specific for the melanoma antigen pMel (see material and methods) comprising the mutated chains as described above. The transduced TCR expression was assayed through cytofluorimetric analysis (FIG. 5). The percentage of Vb13 positive cells in the untransduced splenocytes (6.4%) correspond to the normal TCR variable repertoire, and the increase in this percentage up to 25% in the transduced cells means that the cells have been successfully infected and express the specific therapeutic TCR. The cytotoxic activity of the transduced cells against target cells loaded with the gp100₂₅₋₃₃ peptide KVPRNQDWL (SEQ ID NO: 13) corresponding to the antigenic determinant pMel was measured through calcein release assay (FIG. 6). FIG. 6 shows the specific cytotoxic activity arising from the transduction with the TCR.

Fret Analysis was Performed on Splenocytes Infected with TCR OT-I which Recognizes the OVA 257-263 (SIINKFEL, SEQ ID NO: 32) Peptide.

FRET is a photo physical process involving a non-radioactive transfer of energy from an initially excited donor to a fluorescent acceptor, which in turns emits light at longer wavelength (Clegg R M, Murchie A I, Zechel A, Carlberg C, Diekmann S, Lilley D M. Biochemistry. 1992 May 26;31(20):4846-56). One of the main limit of this method is the spectral bleed-through contamination resulting from fluorescence overlap between the donor and the acceptor. To overcome this limit, in the present study, FRET analysis was performed by using acceptor photo bleaching technique. This represents a good tool to measure protein-protein interaction, without the problem that the acceptor emission bleed-through into the donor channel. The emission separation is achieved by choosing an appropriate optical band-pass filter, which transmits only the specific part of a fluorophore spectrum. Splenocytes transduced with wild type or mutated TCR were stained with monoclonal antibody specific for the murine variable region Vα2 conjugated with CF568, as donor, and monoclonal antibody specific for the murine variable region Vβ5 conjugated with CF647, as acceptor. The data shown in FIG. 7 demonstrate a higher FRET SIGNAL with splenocytes infected with mutated TCR according to the invention, comprising SEQ ID NO:2 in the alpha chain and SEQ ID NO: 4 in the beta chain. The correct pairing of mutated alpha and beta chains was also assayed by FLIM analysis. The results illustrated in FIG. 8 confirm the FRET data and exclude that FRET signals were caused by the formation of homodimers.

Functional Activity of Transduced Splenocytes

Splenocytes transduced with wild type or mutated TCRs were also analyzed for their ability to recognize target cells. In particular the IFNγ production by transduced splenocytes cocultured with antigen presenting cells pre-pulsed with the OVA peptide was assayed. The results illustrated in FIG. 9 show the higher IFNγ production by CD8+ splenocytes transduced with the retroviral vector encoding for the mutated TCR in comparison to CD8+ T cells transduced with the wt TCR. Moreover, the specificity of the transduced splenocytes was also assayed towards B16 melanoma cells expressing the ovalbumin protein. As shown in FIG. 8 only CD8+ T cells transduced with the mutated TCR were able to produce IFNγ when cocultured with Ovalbumin expressing B16 melanoma cells. These results demonstrate that the mutations introduced in TM regions of alpha and beta chains confer an improved functional activity of the transduced TCR specificity as a result of an ameliorated pairing ability of alpha and beta mutated TCR chains.

Results

The invention described herein provides an efficient strategy to induce the correct formation of heterodimeric alpha and beta chains of TCR while excluding the formation of unwanted mixed heterodimers or non-functional homodimeric TCRs. The inventors accomplished this by introducing specific mutations in the transmembrane regions of TCR alpha and beta chains, mimicking a key motif that has been demonstrated to possess a number of interesting structural implications in the Ig TMD dimer assembly and, in turn, in the function of BCR.

To this purpose, using molecular modelling the inventors identified three residues in the transmembrane regions of TCR alpha and beta chains which, when replaced with two hydrophobic (phenylalanine) residues and one polar (serine or threonine) could mimic the motif described for the immunoglobulin heavy chains. In addition, on the basis of sequence alignment and 3D modelling of mouse and human transmembrane regions mutated as above, the authors identified single residue functionalities not involved in the assembly of mutated TCR pairing chains but candidate for optimal interaction with CD3 complex molecule (FIG. 11). It was demonstrated that the introduction of an I/M substitution in the human alpha chain significantly improved its expression on the cell surface (FIG. 10). The experiment in FIG. 10 clearly demonstrates that the percentage of CD3 positive cells measured 24 hours after the infection with retrovirus encoding the mutant TCR comprising in the mutated alpha chain the additional I/M mutation is significantly higher than the percentage of CD3 positive cells measured in the experiment with the mutated alpha chain not comprising the I/M mutation.

The introduced phenylalanine residues stabilize two pairs of inter-chain hydrophobic interactions strengthened by an H bond connecting the serine residues. The introduction of reciprocal mutations that sustain the steric and electrostatic environment within the constant domain of TM regions modified complementarity such that almost exclusively the exogenous

TCRs chains pair together. The hydrophobic phenylalanine residues and the polar serine residue have to be introduced into both TCR chains to provide the inter-helical interactions. Mutant chains are unable to associate with endogenous wild type chains, thereby the formation of hybrid TCRs is avoided.

Since it is known that both alpha and beta TCR chains are necessary to associate with the CD3 complex the inventors hypothesized that unwanted homodimers cannot be exported to the cell surface and therefore would not interfere with the present procedure The presence of homodimers has never been reported, probably because the TCR CD3 complex is expressed on the surface upon correct folding, and this happens only when the heterodimer is associated to TCR.

As a consequence, the inventors reasoned that, once the designed mutations were appropriately introduced into TCR-chains-expressing vectors, it could be possible to monitor if and when the correct association of functionally active TCR complexes was achieved. For this purpose it was used a previously described system based on transfections in hybridoma T cells deprived of endogenous TCR chains followed by antibody staining of transfected cell surfaces and cytofluorimetric analysis [8].

More specifically, by using all possible pairwise combinations of equal amounts of wild type and/or mutated alpha and/or mutated beta chain vector DNA, it was possible to:
Compare mock transfections with transfections of wild type alpha and beta chains in homo or hetero combinations to obtain information on correctness of the procedure and baseline and reference values for the other experiments. Baseline values correspond to CD3 expression in negative samples (mean of fluorescence intensity about 200). Reference value is the CD3 expression with wt TCR.
Transfect mutated alpha and beta chains to ascertain the ability to form correct heterodimers. Furthermore functional TCR formation can be assessed in the appropriate cellular background by monitoring IL-2 production after TCR activation.
Transfect mutant alpha alone or mutant beta alone to monitor unwanted homodimers formation.
Transfect wild type and mutant chains of opposite alpha/beta types to monitor unwanted mixed WT-Mutant heterodimers.

Experiment design and results output are summarized in the following Table 1.

TABLE 1
Output of the expected results of transfection experiment

| | Alpha WT | Alpha Mut | Beta WT | Beta Mut |
|---|---|---|---|---|
| Alpha WT | Provides Baseline | | | |
| | values | | | |
| Alpha MUT | Assessment of | Assessment of | | |
| | unwanted mixed | unwanted (non- | | |
| | (non-functional) | functional) | | |
| | homodimers | homodimers | | |
| Beta WT | Provides | Assessment of | Provides Baseline | |
| | Reference values | unwanted mixed | values | |
| | | heterodimeric | | |
| | | TCRs | | |
| Beta Mut | Assessment of | Assessment of | Assessment of | Assessment of |
| | unwanted mixed | correct TCR | unwanted | unwanted |
| | heterodimeric | formation | mixed (non- | homodimers |
| | TCRs | | functional) | |
| | | | homodimers | |

The efficacy of the present methodology was further demonstrated by the analysis of the functional activity of isolated T cells from splenocytes of C57BL/6 mice (Charles River, Lecco, Italy) and transduced with a retroviral vector encoding for the mutated TCR chains.

Further proofs of concept were collected on the functional activity of mutated TCR according to the invention. In particular, using FRET analysis the inventors demonstrated the preferential pairing of mutated TCR. These results were confirmed by FLIM analysis, which show at single cell level the consistency of the preferential pairing of alpha and beta mutated chains in respect to wild type chains.

Moreover, the functional activity of mouse splenocytes transduced with the mutated chains and co-cultured with target cells displaying an antigen specifically recognized by the transduced TCR was analyzed in vitro by measuring IFNγ production.

### REFERENCES

1) Kochenderfer J N and Rosenberg S A (2013). Nat Rev Clin Oncol 5: 267-76

2) Bendle G M, et al. (2010) Nat Med 5: 565-70

3) Merhavi-Shoham E et al. (2012). Semin Cancer Biol 22(1):14-22

4) Haga-Friedman A, Horovitz-Fried M, Cohen C J (2012) J Immunol 188(11):5538-46

5) O'Shea E, Lumb K, Kim P. (1993) Current Biology 3(10):658-667

6) Chang H C et al. (1994) Proc Natl Acad Sci U S A 91(24):11408-12.

7) Varriale S, et al. (2010) Mol Phylogenet Evol 57: 1238-44

8) Caivano A et al. (2011) J Immunol Methods 255(1-2):125-34.

9) Kerkar S P et al. (2011) J Immunother 34(4): 343-52

10) Paul W E, Fundamental Immunology Lippincot Raven publisher

11) Sartorius, R., et al. (2008) J of Immunol 180:3719-3728.

12) Yang Sl, et al. (2008) Gene Ther. November; 15(21):1411-23.

13) Kerry S. Campbell et al. (1994) Sem Immunol (6):393-410.

14) Schodin B. A., Tsomides T. J., Kranz D. M. (1996). Immunity 5 137-146

15) Uckert, W., Schumacher T. N. (2009) Cancer Immunol. Immunither. 58: 809-822

16) Varela-Rohena, A., Molloy P. E., Dunn S. M. et al (2008) Nat. Med. 14:1390-1395.

17) Sartorius R, et al. (2011) Eur. J. Immunol. 41 (9):2573-84.

18) Cohen C J et al. (2007) Cancer Res; 67:3898-903.

19) Sommermeyer D., Uckert W. (2010) J. Immunol. 184:6223-31.

20) Sebestyen Z., Schooten E., Sals T., et al. (2008) J. Immunol. 180: 7736-7746.

21) Chung S et al Proc Natl Acad Sci USA (1994), 91:12654-8.

22) Okamoto S et al. (2009) Cancer Res; 69:9003-11.

23) Bendle G M et al., (2010) Nat Med;16(5):565-70.

24) Heemskerk, M. H et al (2003) Blood 102:3530-3540.

25) Pascolo, S et al. (1997) J. Exp. Med. 185 (12) 2043-2051.

26) Kerkar S P, et al. (2011) J Immunother. May; 34(4):343-52.

1. A T Cell Receptor (TCR) comprising at least one of an alpha chain and a beta chain;
wherein the alpha chain comprises a transmembrane region comprising SEQ ID NO:1, SEQ ID NO:2, SEQ ID NO:14, SEQ ID NO:15, OR SEQ ID NO:16; and
wherein the beta chain comprises a transmembrane region comprising SEQ ID NO:3, SEQ ID NO:4, SEQ ID NO:17, SEQ ID NO:18, SEQ ID NO:19, OR SEQ ID NO:20, wherein said transmembrane region is characterized by mutations in the amino acid positions 8 and 12 with an hydrophobic amino acid residue, and in position 15 with a polar amino acid residue.

2. The T Cell Receptor (TCR) alpha or beta chain according to claim 1, wherein the hydrophobic amino acid residue is phenylalanine and the polar amino acid residue is serine or threonine.

3. The T Cell Receptor (TCR) alpha chain according to claim 1, further comprising the amino acid methionine in position 2.

4. The T Cell Receptor (TCR) alpha or beta chain according to claim 1, wherein the polar aminoacid is serine.

5. (canceled)

6. (canceled)

7. (canceled)

8. (canceled)

9. (canceled)

10. (canceled)

11. The T Cell Receptor (TCR) according to claim 1, wherein the TCR is specific for an antigen selected from the group consisting of: a tumor cell antigen, a tumor cell associated antigen, and a pathogenic agent.

12. The T Cell Receptor (TCR) according to claim 11 wherein the tumor cell antigen or the tumor cell associated antigen is selected from an antigen of a hematological malignancy or of a solid tumor.

13. The T Cell Receptor (TCR) according to claim 11 wherein the antigen is selected from the group consisting of: influenza virus, measles and respiratory syncytial virus, dengue virus, human immunodeficiency virus, human hepatitis virus, herpes virus, papilloma virus, Plasmodium falciparum protozoa, or a mycobacteria.

14. The T Cell Receptor (TCR) according to claim 1, further comprising associated with a detectable label, a therapeutic agent, a PK modifying moiety or a combination thereof.

15. A TCR complex comprising at least two TCRs according to claim 1.

16. A nucleic acid coding for the T Cell Receptor (TCR) alpha and/or beta chain according to claim 1.

17. A recombinant expression vector comprising the nucleic acid according to claim 16, wherein said vector is a retroviral or lentiviral vector.

18. A host cell comprising the nucleic acid claim 16.

19. A method to generate cells expressing a T Cell Receptor (TCR), the method comprising the following steps:
activating a population of lymphocytes obtained from peripheral blood of a subject;
isolating the T cells from said population;
transducing or transfecting the isolated T cells with a nucleic acid coding for a TCR comprising at least one of an alpha chain and a beta chain;
wherein the alpha chain comprises a transmembrane region comprising SEQ ID NO:1, SEQ ID NO:2 SEQ ID NO:14, SEQ ID NO:15, or SEQ ID NO:16; and
wherein the beta chain comprises a transmembrane region comprising SEQ ID NO:3, SEQ ID NO:4, SEQ ID NO:17 SEQ ID NO:18, SEQ ID NO:19, or SEQ ID NO:20, wherein said transmembrane region is characterized by mutations in the amino acid positions 8 and 12 with an hydrophobic amino acid residue, and in position 15 with a polar amino acid residue.

20. A cell expressing the T Cell Receptor (TCR) prepared according to the method recited in claim 19.

21. A pharmaceutical composition comprising the nucleic acid according to claim 16, and further comprising a pharmaceutically acceptable vehicle and/or adjuvant.

22. (canceled)

23. (canceled)

24. (canceled)

25. A method for the treatment and/or prevention of a hematological tumor, a solid tumor or an infective disease comprising administering to a subject the T Cell Receptor (TCR) of claim 1.

26. A method of detecting at least one of a hematological malignant cell, a solid tumor cell or an infected cell, the method comprising:
providing a T Cell Receptor (TCR) comprising at least one of an alpha chain and a beta chain;
wherein the alpha chain comprises a transmembrane region comprising SEQ ID NO:1, SEQ ID NO:2, SEQ ID NO:14, SEQ ID NO:15, or SEQ ID NO:16;
wherein the beta chain comprises a transmembrane region comprising SEQ ID NO:3, SEQ ID NO:4, SEQ ID NO:17, SEQ ID NO:18, SEQ ID NO:19, or SEQ ID NO:20, wherein said transmembrane region is characterized by mutations in the amino acid positions 8 and 12 with an hydrophobic amino acid residue, and in position 15 with a polar amino acid residue;
contacting a sample comprising the hematological malignant cell, solid tumor cell or the infected cell with the TCR, thereby forming a complex; and
detecting the complex, wherein detection of the complex is indicative of the presence of an hematological malignancy, a solid tumor or an infective disease.

27. The T Cell Receptor (TCR) according to claim 11, wherein the TCR is specific for pathogenic agent derived from a virus, bacteria, protozoa, or parasite.

28. The TCR according to claim 12, wherein the tumor cell antigen is selected from a multiple myeloma, melanoma, lung tumor, endometrial tumor, glioma, lymphoma, leukemia, or prostate tumor.

29. A host cell according to claim 18, wherein said host cell is a T lymphocyte.
